(19) United States
(12) Patent Application Publication
(10) Pub. No.: US 2010/0280031 A1
(43) Pub. Date: Nov. 4, 2010

(54) LIPID FORMULATION OF APOPTOSIS PROMOTER

(76) Inventors: Paul David, Lindenhurst, IL (US); Michael G. Fickes, Evanston, IL (US); Cristina M. Fischer, Wadsworth, IL (US); Anthony R. Haight, Wadsworth, IL (US); Katherine Heemstra, Chicago, IL (US); Kennan Marsh, Lake Forest, IL (US); Peter Mayer, Libertyville, IL (US); Vitaly Rubin, Niles, IL (US); Yeshwant D. Sanzgiri, Gurnee, IL (US); Eric A. Schmitt, Libertyville, IL (US); Ping Tong, Libertyville, IL (US); Deliang Zhou, Vernon Hills, IL (US)

Correspondence Address: Harness Dickey & Pierce, PLC, 7700 Bonhomme, Suite 400, Clayton, MO, 63105, US

(21) Appl. No.: 12/770,122

(22) Filed: Apr. 29, 2010

Related U.S. Application Data
(60) Provisional application No. 61/174,245, filed on Apr. 30, 2009.

(51) Int. Cl. A61K 31/5377; A61K 31/4965; A61P 35/00; A61P 35/02

(52) U.S. Cl. 514/235.8; 514/255.03

(57) ABSTRACT

An orally deliverable pharmaceutical composition comprises a drug-carrier system having a Bcl-2 family protein inhibitory compound, e.g., ABT-263, in solution in a substantially non-aqueous carrier that comprises at least one phospholipid and a pharmaceutically acceptable solubilizing agent. The composition is suitable for oral administration to a subject in need thereof for treatment of a disease characterized by overexpression of one or more anti-apoptotic Bcl-2 family proteins, for example cancer.

### CROSS-REFERENCE TO RELATED APPLICATIONS

This application claims priority benefit of U.S. Provisional Application Ser. No. 61/174,245 filed on Apr. 30, 2009.

Cross-reference is made to the following co-filed U.S. applications containing subject matter related to the present application: Ser. No. 12/______ titled “Salt of ABT-263 and solid-state forms thereof”, which claims priority benefit of U.S. provisional application Ser. No. 61/174,274 filed on Apr. 30, 2009; and Ser. No. 12/______ titled “Stabilized lipid formulation of apoptosis promoter”, which claims priority benefit of U.S. provisional application Ser. No. 61/174,299 filed on Apr. 30, 2009 and Ser. No. 61/289,254 filed on Dec. 22, 2009.

The entire disclosure of each of the above applications is incorporated herein by reference.

### FIELD OF THE INVENTION

The present invention relates to pharmaceutical compositions comprising an apoptosis-promoting agent, and to methods of use thereof for treating diseases characterized by overexpression of anti-apoptotic Bcl-2 family proteins. More particularly the invention relates to such compositions exhibiting improved oral bioavailability of the apoptosis-promoting agent and to oral dosage regimens for administration of such a composition to a subject in need thereof.

### BACKGROUND OF THE INVENTION

Evasion of apoptosis is a hallmark of cancer (Hanahan & Weinberg (2000) Cell 100:57-70). Cancer cells must overcome a continual bombardment by cellular stresses such as DNA damage, oncogene activation, aberrant cell cycle progression and harsh microenvironments that would cause normal cells to undergo apoptosis. One of the primary means by which cancer cells evade apoptosis is by up-regulation of anti-apoptotic proteins of the Bcl-2 family.

Compounds that occupy the BH3 binding groove of Bcl-2 proteins have been described, for example by Bruncko et al. (2007) J. Med. Chem. 50:641-662. These compounds have included N-(4-(4-((4′-chloro-(1,1′-biphenyl)-2-yl)methyl)piperazin-1-yl) benzoyl)-4-(((1R)-3-(dimethylamino)-1-((phenylsulfanyl)methyl)propyl)amino)-3-nitrobenzene-sulfonamide, otherwise known as ABT-737, which has the formula:

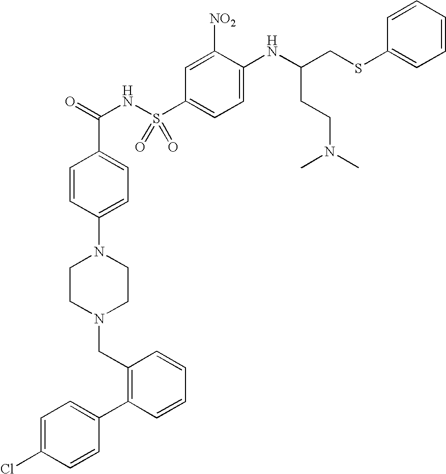

ABT-737 binds with high affinity (<1 nM) to proteins of the Bcl-2 family (specifically Bcl-2, Bcl-X_{L} and Bcl-w). It exhibits single-agent activity against small-cell lung cancer (SCLC) and lymphoid malignancies, and potentiates pro-apoptotic effects of other chemotherapeutic agents. ABT-737 and related compounds, and methods to make such compounds, are disclosed in U.S. Patent Application Publication No. 2007/0072860 of Bruncko et al.

More recently, a further series of compounds has been identified having high binding affinity to Bcl-2 family proteins. These compounds, and methods to make them, are disclosed in U.S. Patent Application Publication No. 2007/0027135 of Bruncko et al. (herein “the '135 publication”), incorporated by reference herein in its entirety, and can be seen from their formula (Formula I below) to be structurally related to ABT-737.

In compounds of Formula I:

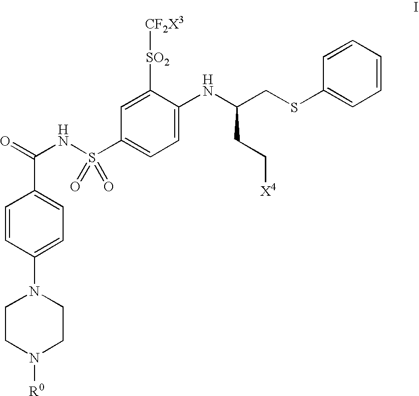

X³ is chloro or fluoro; and
(1) X⁴ is azepan-1-yl, morpholin-4-yl, 1,4-oxazepan-4-yl, pyrrolidin-1-yl, N(CH₃)₂, N(CH₃)(CH(CH₃)₂), 7-azabicyclo[2.2.1]heptan-1-yl or 2-oxa-5-azabicyclo[2.2.1]hept-5-yl; and R⁰ is

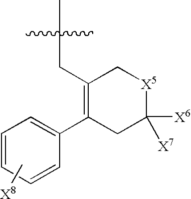

where
X⁵ is CH₂, C(CH₃)₂ or CH₂CH₂;
X⁶ and X⁷ are both hydrogen or both methyl; and
X⁸ is fluoro, chloro, bromo or iodo; or
(2) X⁴ is azepan-1-yl, morpholin-4-yl, pyrrolidin-1-yl, N(CH₃)(CH(CH₃)₂) or 7-azabicyclo[2.2.1]heptan-1-yl; and R⁰ is

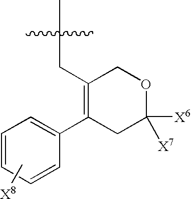

where X⁶, X⁷ and X⁸ are as above; or
(3) X⁴ is morpholin-4-yl or N(CH₃)₂; and R⁰ is

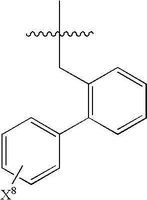

where X⁸ is as above.

The '135 publication states that while inhibitors of Bcl-2 family proteins previously known may have either potent cellular efficacy or high systemic exposure after oral administration, they do not possess both properties. A typical measure of cellular efficacy of a compound is the concentration eliciting 50% cellular effect (EC₅₀). A typical measure of systemic exposure after oral administration of a compound is the area under the curve (AUC) resulting from graphing plasma concentration of the compound versus time from oral administration. Previously known compounds, it is stated in the '135 publication, have a low AUC/EC₅₀ ratio, meaning that they are not orally efficacious. Compounds of Formula I, by contrast, are stated to demonstrate enhanced properties with respect to cellular efficacy and systemic exposure after oral administration, resulting in a AUC/EC₅₀ ratio significantly higher than that of previously known compounds.

One compound, identified as “Example 1” in the '135 publication, is N-(4-(4-((2-(4-chlorophenyl)-5,5-dimethyl-1-cyclohex-1-en-1-yl)methyl)piperazin-1-yl)benzoyl)-4-4(((1R)-3-(morpholin-4-yl)-1-((phenylsulfanyl)methyl)propyl)amino-3-((trifluoromethyl)sulfonyl)benzenesulfonamide, otherwise known as ABT-263. This compound has a molecular weight of 974.6 g/mol and has the formula:

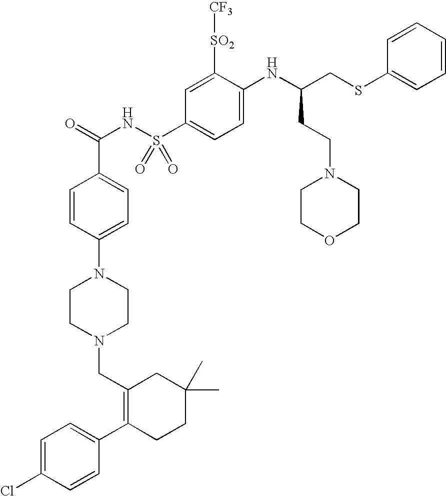

ABT-263 binds with high affinity (<1 nM) to Bcl-2 and Bcl-X_{L} and is believed to have similarly high affinity for Bcl-w. Its AUC/EC₅₀ ratio is reported in the '135 publication as 56, more than an order of magnitude greater than that reported for ABT-737 (4.5). For determination of AUC according to the '135 publication, each compound was administered to rats in a single 5 mg/kg dose by oral gavage as a 2 mg/ml solution in a vehicle of 10% DMSO (dimethyl sulfoxide) in PEG-400 (polyethylene glycol of average molecular weight about 400).

Oral bioavailability (as expressed, for example, by AUC after oral administration as a percentage of AUC after intravenous administration) is not reported in the '135 publication, but can be concluded therefrom to be substantially greater for ABT-263 than for ABT-737. However, further improvement in oral bioavailability would be advantageous. Various solutions to the challenge of low oral bioavailability have been proposed in the art. For example, U.S. Pat. No. 5,645,856 to Lacy et al. proposes formulating a hydrophobic drug with (a) an oil, (b) a hydrophilic surfactant and (c) a lipophilic surfactant that substantially reduces an inhibitory effect of the hydrophilic surfactant on in vivo lipolysis of the oil, such lipolysis being said to be a factor promoting bioavailability of the drug. Among numerous classes of hydrophilic surfactants listed are phospholipids such as lecithins.

U.S. Pat. No. 6,267,985 to Chen & Patel is directed, inter alia, to a pharmaceutical composition comprising (a) a triglyceride, (b) a carrier comprising at least two surfactants, one of which is hydrophilic, and (c) a therapeutic agent capable of being solubilized in the triglyceride, the carrier or both. It is specified therein that the triglyceride and the surfactants must be present in amounts providing a clear aqueous dispersion when the composition is mixed with an aqueous solution under defined conditions. Among extensive separate lists of exemplary ingredients, mention is made of “glyceryl tricaprylate/caprate” as a triglyceride, and phospholipids including phosphatidyl-choline as surfactants.

U.S. Pat. No. 6,451,339 to Patel & Chen mentions disadvantages of presence of triglycerides in such compositions, and proposes otherwise similar compositions that are substantially free of triglycerides, but that likewise provide clear aqueous dispersions.

U.S. Pat. No. 6,309,663 to Patel & Chen proposes pharmaceutical compositions comprising a combination of surfactants said to enhance bioabsorption of a hydrophilic therapeutic agent. Phospholipids such as phosphatidylcholine are again listed among exemplary surfactants.

U.S. Pat. No. 6,464,987 to Fanara et al. proposes a fluid pharmaceutical composition comprising an active substance, 3% to 55% by weight of phospholipid, 16% to 72% by weight of solvent, and 4% to 52% by weight of fatty acid. Compositions comprising Phosal 50 PG™ (primarily comprising phosphatidylcholine and propylene glycol), in some cases together with Phosal 53 MCT™ (primarily comprising phosphatidylcholine and medium chain triglycerides), are specifically exemplified. Such compositions are said to have the property of gelling instantaneously in presence of an aqueous phase and to allow controlled release of the active substance.

U.S. Pat. No. 5,538,737 to Leonard et al. proposes a capsule containing a water-in-oil emulsion wherein a water-soluble drug salt is dissolved in the water phase of the emulsion and wherein the oil phase comprises an oil and an emulsifying agent. Among oils mentioned are medium chain triglycerides; among emulsifying agents mentioned are phospholipids such as phosphatidylcholine. Phosal 53 MCT™, which contains phosphatidylcholine and medium chain triglycerides, is reportedly used according to various examples therein.

U.S. Pat. No. 5,536,729 to Waranis & Leonard proposes an oral formulation comprising rapamycin, at a concentration of about 0.1 to about 50 mg/ml, in a carrier comprising a phospholipid solution. It is stated therein that a preferred formulation can be made using Phosal 50 PG™ as the phospholipid solution. An alternative phospholipid solution mentioned is Phosal 50 MCT™.

U.S. Pat. No. 5,559,121 to Harrison et al. proposes an oral formulation comprising rapamycin, at a concentration of about 0.1 to about 100 mg/ml, in a carrier comprising N,N-dimethylacetamide and a phospholipid solution. Examples of the more preferred embodiments are shown to be prepared using Phosal 50 PG™. An alternative phospholipid solution mentioned is Phosal 50 MCT™.

U.S. Patent Application Publication No. 2007/0104780 of Lipari et al. discloses that a small-molecule drug (defined therein as having molecular weight, excluding counterions in the case of salts, not greater than about 750 g/mol, typically not greater than about 500 g/mol) having low water solubility can be formulated as a solution in a substantially non-aqueous carrier comprising at least one phospholipid and a pharmaceutically acceptable solubilizing agent. The solution, when mixed with an aqueous phase, is said to form a non-gelling, substantially non-transparent liquid dispersion. Illustratively, formulations of N-(4-(3-amino-1H-indazol-4-yl)phenyl)-N′-(2-fluoro-5-methylphenyl)urea (the protein tyrosine kinase inhibitor ABT-869) comprising Phosal 53 MCT™ and other ingredients are described therein.

A particular type of disease for which improved therapies are needed is non-Hodgkin's lymphoma (NHL). NHL is the sixth most prevalent type of new cancer in the U.S. and occurs primarily in patients 60-70 years of age. NHL is not a single disease but a family of related diseases, which are classified on the basis of several characteristics including clinical attributes and histology.

One method of classification places different histological subtypes into two major categories based on natural history of the disease, i.e., whether the disease is indolent or aggressive. In general, indolent subtypes grow slowly and are generally incurable, whereas aggressive subtypes grow rapidly and are potentially curable. Follicular lymphomas are the most common indolent subtype, and diffuse large-cell lymphomas constitute the most common aggressive subtype. The oncoprotein Bcl-2 was originally described in non-Hodgkin's B-cell lymphoma.

Treatment of follicular lymphoma typically consists of biologically-based or combination chemotherapy. Combination therapy with rituximab, cyclophosphamide, doxorubicin, vincristine and prednisone (R-CHOP) is routinely used, as is combination therapy with rituximab, cyclophosphamide, vincristine and prednisone (RCVP). Single-agent therapy with rituximab (targeting CD20, a phosphoprotein uniformly expressed on the surface of B-cells) or fludarabine is also used. Addition of rituximab to chemotherapy regimens can provide improved response rate and increased progression-free survival.

Radioimmunotherapy agents, high-dose chemotherapy and stem cell transplants can be used to treat refractory or relapsed non-Hodgkin's lymphoma. Currently, there is not an approved treatment regimen that produces a cure, and current guidelines recommend that patients be treated in the context of a clinical trial, even in a first-line setting.

First-line treatment of patients with aggressive large B-cell lymphoma typically consists of rituximab, cyclophosphamide, doxorubicin, vincristine and prednisone (R-CHOP), or dose-adjusted etoposide, prednisone, vincristine, cyclophosphamide, doxorubicin and rituximab (DA-EPOCH-R).

Most lymphomas respond initially to any one of these therapies, but tumors typically recur and eventually become refractory. As the number of regimens patients receive increases, the more chemotherapy-resistant the disease becomes. Average response to first-line therapy is approximately 75%, 60% to second-line, 50% to third-line, and about 35-40% to fourth-line therapy. Response rates approaching 20% with a single agent in a multiple relapsed setting are considered positive and warrant further study.

Current chemotherapeutic agents elicit their antitumor response by inducing apoptosis through a variety of mechanisms. However, many tumors ultimately become resistant to these agents. Bcl-2 and Bcl-X_{L} have been shown to confer chemotherapy resistance in short-term survival assays in vitro and, more recently, in vivo. This suggests that if improved therapies aimed at suppressing the function of Bcl-2 and Bcl-X_{L} can be developed, such chemotherapy-resistance could be successfully overcome.

Apoptosis-promoting drugs that target Bcl-2 family proteins such as Bcl-2 and Bcl-X_{L} are best administered according to a regimen that provides continual, for example daily, replenishment of the plasma concentration, to maintain the concentration in a therapeutically effective range. This can be achieved by daily parenteral, e.g., intravenous (i.v.) or intraperitoneal (i.p.) administration. However, daily parenteral administration is often not practical in a clinical setting, particularly for outpatients. To enhance clinical utility of an apoptosis-promoting agent, for example as a chemotherapeutic in cancer patients, a dosage form with good oral bioavailability would be highly desirable. Such a dosage form, and a regimen for oral administration thereof, would represent an important advance in treatment of many types of cancer, including non-Hodgkin's lymphoma, and would more readily enable combination therapies with other chemotherapeutics.

### SUMMARY OF THE INVENTION

It has been found that oral bioavailability of the lead Bcl-2 protein family inhibitor ABT-737 is not substantially affected by the carrier system in which it is formulated. Despite this discouraging result, the present inventors have continued the search for a Bcl-2 protein family inhibitory composition and have discovered that ABT-263, when formulated in a lipid carrier system comprising a phospholipid and a solubilizing agent, exhibits unexpectedly high oral bioavailability by comparison with compositions described, for example, in the above-cited '135 publication.

There is accordingly provided an orally deliverable pharmaceutical composition comprising a drug-carrier system that comprises a compound of Formula I:

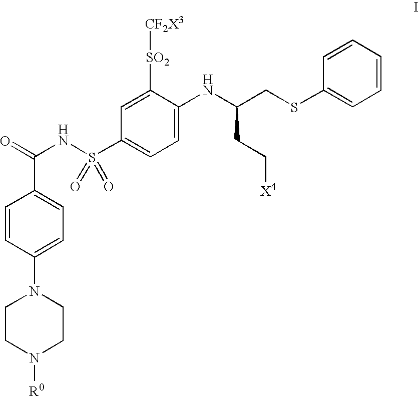

where X³ is chloro or fluoro; and
(1) X⁴ is azepan-1-yl, morpholin-4-yl, 1,4-oxazepan-4-yl, pyrrolidin-1-yl, N(CH₃)₂, N(CH₃)(CH(CH₃)₂), 7-azabicyclo[2.2.1]heptan-1-yl or 2-oxa-5-azabicyclo[2.2.1]hept-5-yl; and R⁰ is

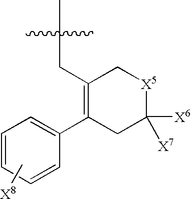

where
X⁵ is CH₂, C(CH₃)₂ or CH₂CH₂;
X⁶ and X⁷ are both hydrogen or both methyl; and
X⁸ is fluoro, chloro, bromo or iodo; or
(2) X⁴ is azepan-1-yl, morpholin-4-yl, pyrrolidin-1-yl, N(CH₃)(CH(CH₃)₂) or 7-azabicyclo[2.2.1]heptan-1-yl; and R⁰ is

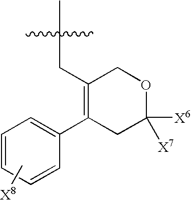

where X⁶, X⁷ and X⁸ are as above; or
(3) X⁴ is morpholin-4-yl or N(CH₃)₂; and R⁰ is

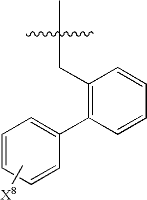

where X⁸ is as above;
   or a pharmaceutically acceptable salt, prodrug, salt of a prodrug or metabolite thereof; in solution in a substantially non-aqueous carrier that comprises a phospholipid component and a pharmaceutically acceptable solubilizing component; wherein the carrier comprises zero to about 25% by weight ethanol.

There is further provided an orally deliverable pharmaceutical composition comprising a drug-carrier system that comprises the compound N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino-3-((trifluoromethyl)sulfonyl)benzenesulfonamide (ABT-263) or a salt, prodrug, salt of a prodrug or metabolite thereof; in solution in a substantially non-aqueous carrier that comprises a phospholipid component and a pharmaceutically acceptable solubilizing component; wherein the carrier comprises zero to about 25% by weight ethanol. In a more particular embodiment, the compound is ABT-263 free base or ABT-263 bis-hydrochloride salt (ABT-263 bis-HCl).

There is further provided a method for treating a disease characterized by apoptotic dysfunction and/or overexpression of an anti-apoptotic Bcl-2 family protein, comprising orally administering to a subject having the disease a therapeutically effective amount of a composition as described above. Examples of such a disease include many neoplastic diseases including cancers. A specific illustrative type of cancer that can be treated according to the present method is non-Hodgkin's lymphoma. Another specific illustrative type of cancer that can be treated according to the present method is chronic lymphocytic leukemia. Yet another specific illustrative type of cancer that can be treated according to the present method is acute lymphocytic leukemia, for example in a pediatric patient.

There is still further provided a method for maintaining in bloodstream of a human cancer patient, for example a patient having non-Hodgkin's lymphoma, chronic lymphocytic leukemia or acute lymphocytic leukemia, a therapeutically effective plasma concentration of ABT-263 and/or one or more metabolites thereof, comprising administering to the subject a pharmaceutical composition comprising a drug-carrier system that comprises ABT-263 or a pharmaceutically acceptable salt, prodrug, salt of a prodrug or metabolite thereof (for example ABT-263 free base or ABT-263 bis-HCl), in solution in a substantially non-aqueous carrier that comprises a phospholipid component and a pharmaceutically acceptable solubilizing component, wherein the carrier comprises zero to about 25% by weight ethanol, in a dosage amount equivalent to about 50 to about 500 mg ABT-263 per day, at an average dosage interval of about 3 hours to about 7 days.

Additional embodiments of the invention, including more particular aspects of those provided above, will be found in, or will be evident from, the detailed description that follows.

### BRIEF DESCRIPTION OF THE DRAWINGS

FIG. 1 is a graphical representation of human clinical single-dose pharmacokinetic (PK) data under fasting and non-fasting conditions, showing dose-proportionality of PK parameters AUC₀₋₂₄ and Cₘₐₓ for ABT-263 administered in a composition of the present invention as described in Example 9.

FIG. 2 is a graphical representation of ABT-263 plasma concentrations in a human clinical study following a single 315 mg dose (fasting and non-fasting) and at steady state following 315 mg daily doses (non-fasting), of ABT-263 administered in a composition of the present invention as described in Example 9.

### DETAILED DESCRIPTION

A “drug-carrier system” herein comprises a carrier having at least one drug homogeneously distributed therein. In compositions of the present invention the drug is in solution in the carrier, and, in some embodiments, the drug-carrier system constitutes essentially the entire composition. In other embodiments, the drug-carrier system is encapsulated within a capsule shell that is suitable for oral administration; in such embodiments the composition comprises the drug-carrier system and the capsule shell.

The carrier and the drug-carrier system are typically liquid, but in some embodiments the carrier and/or the drug-carrier system can be solid or semi-solid. For example, a drug-carrier system can illustratively be prepared by dissolving the drug in a carrier at a temperature above the melting or flow point of the carrier, and cooling the resulting solution to a temperature below the melting or flow point to provide a solid drug-carrier system. Alternatively or in addition, the carrier can comprise a solid substrate wherein or whereon a solution of the drug as described herein is adsorbed.

A composition of the invention is “orally deliverable”, i.e., adapted for oral administration; however, such a composition can be useful for delivery of the drug to a subject in need thereof by other routes of administration, including without limitation parenteral, sublingual, buccal, intranasal, pulmonary, topical, transdermal, intradermal, ocular, otic, rectal, vaginal, intragastric, intracranial, intrasynovial and intra-articular routes.

The terms “oral administration” and “orally administered” herein refer to administration to a subject per os (p.o.), that is, administration wherein the composition is immediately swallowed, for example with the aid of a suitable volume of water or other potable liquid. “Oral administration” is distinguished herein from intraoral administration, e.g., sublingual or buccal administration or topical administration to intraoral tissues such as periodontal tissues, that does not involve immediate swallowing of the composition.

Therapeutically active compounds, including salts, prodrugs, salts of prodrugs and metabolites thereof, useful herein typically have low solubility in water, for example less than about 100 μg/ml, in most cases less than about 30 μg/ml. The present invention can be especially advantageous for drugs that are essentially insoluble in water, i.e., having a solubility of less than about 10 μg/ml. It will be recognized that aqueous solubility of many compounds is pH-dependent; in the case of such compounds the solubility of interest herein is at a physiologically relevant pH, for example a pH of about 1 to about 8. Thus, in various embodiments, the drug has a solubility in water, at least at one point in a pH range from about 1 to about 8, of less than about 100 μg/ml, for example less than about 30 μg/ml, or less than about 10 μg/ml. Illustratively, ABT-263 has a solubility in water at pH 2 of less than 4 μg/ml.

In one embodiment, the composition comprises a compound of Formula I as defined above, or a pharmaceutically acceptable salt, prodrug, salt of a prodrug or metabolite of such a compound.

In a further embodiment, the compound has Formula I where X³ is fluoro.

In a still further embodiment, the compound has Formula I where X⁴ is morpholin-4-yl.

In a still further embodiment, the compound has Formula I where R⁰ is

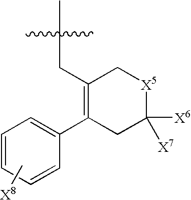

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

In a still further embodiment, the compound has Formula I where R⁰ is

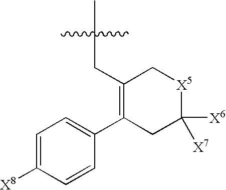

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

In a still further embodiment, the compound has Formula I where X³ is fluoro and X⁴ is morpholin-4-yl.

In a still further embodiment, the compound has Formula I where X³ is fluoro and R⁰ is

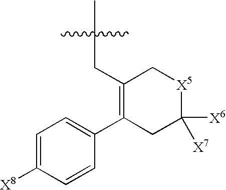

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

In a still further embodiment, the compound has Formula I where X⁴ is morpholin-4-yl and R⁰ is

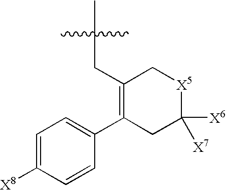

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

In a still further embodiment, the compound has Formula I where X³ is fluoro, X⁴ is morpholin-4-yl and R⁰ is

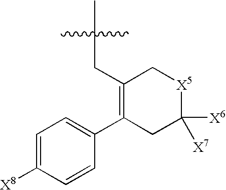

where X⁵ is O, CH₂, C(CH₃)₂ or CH₂CH₂; X⁶ and X⁷ are both hydrogen or both methyl; and X⁸ is fluoro, chloro, bromo or iodo. Illustratively according to this embodiment X⁵ can be CH₂ or C(CH₃)₂ and/or each of X⁶ and X⁷ can be methyl and/or X⁸ can be chloro.

Compounds of Formula I may contain asymmetrically substituted carbon atoms in the R- or S-configuration; such compounds can be present as racemates or in an excess of one configuration over the other, for example in an enantiomeric ratio of at least about 85:15. The compound can be substantially enantiomerically pure, for example having an enantiomeric ratio of at least about 95:5, or in some cases at least about 98:2 or at least about 99:1.

Compounds of Formula I may alternatively or additionally contain carbon-carbon double bonds or carbon-nitrogen double bonds in the Z- or E-configuration, the term “Z” denoting a configuration wherein the larger substituents are on the same side of such a double bond and the term “E” denoting a configuration wherein the larger substituents are on opposite sides of the double bond. The compound can alternatively be present as a mixture of Z- and E-isomers.

Compounds of Formula I may alternatively or additionally exist as tautomers or equilibrium mixtures thereof wherein a proton shifts from one atom to another. Examples of tautomers illustratively include keto-enol, phenol-keto, oxime-nitroso, nitro-aci, imine-enamine and the like.

In some embodiments, a compound of Formula I is present in the composition in its parent-compound form, alone or together with a salt or prodrug form of the compound.

Compounds of Formula I may form acid addition salts, basic addition salts or zwitterions. Salts of compounds of Formula I can be prepared during isolation or following purification of the compounds. Acid addition salts are those derived from reaction of a compound of Formula I with an acid. For example, salts including the acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifluoroacetate, para-toluenesulfonate and undecanoate salts of a compound of Formula I can be used in a composition of the invention. Basic addition salts including those derived from reaction of a compound with the bicarbonate, carbonate, hydroxide or phosphate of cations such as lithium, sodium, potassium, calcium and magnesium can likewise be used.

A compound of Formula I typically has more than one protonatable nitrogen atom and is consequently capable of forming acid addition salts with more than one, for example about 1.2 to about 2, about 1.5 to about 2 or about 1.8 to about 2, equivalents of acid per equivalent of the compound.

ABT-263 can likewise form acid addition salts, basic addition salts or zwitterions. Salts of ABT-263 can be prepared during isolation or following purification of the compound. Acid addition salts derived from reaction of ABT-263 with an acid include those listed above. Basic addition salts including those listed above can likewise be used. ABT-263 has at least two protonatable nitrogen atoms and is consequently capable of forming acid addition salts with more than one, for example about 1.2 to about 2, about 1.5 to about 2 or about 1.8 to about 2, equivalents of acid per equivalent of the compound.

Illustratively in the case of ABT-263, bis-salts can be formed including, for example, bis-hydrochloride (bis-HCl) and bis-hydrobromide (bis-HBr) salts.

For example, ABT-263 bis-HCl, which has a molecular weight of 1047.5 g/mol and is represented by the formula

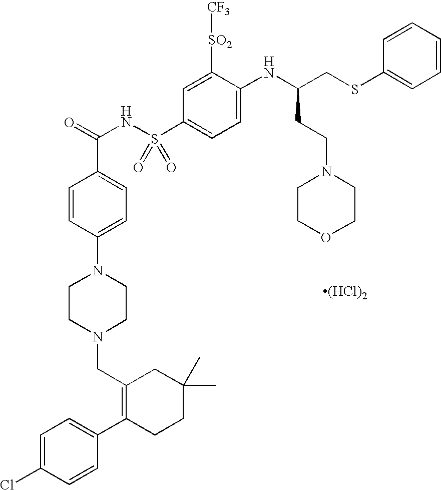

can be prepared by a variety of processes, for example a process that can be outlined as follows.

ABT-263 free base is prepared, illustratively as described in Example 1 of above-cited U.S. Patent Application Publication No. 2007/0027135, the entire disclosure of which is incorporated by reference herein. A suitable weight of ABT-263 free base is dissolved in ethyl acetate. A solution of hydrochloric acid in ethanol (for example about 4.3 kg HCl in 80 g EtOH) is added to the ABT-263 solution in an amount providing at least 2 mol HCl per mol ABT-263 and sufficient EtOH (at least about 20 vol) for crystallization of the resulting ABT-263 bis-HCl salt. The solution is heated to about 45° C. with stirring and seeds are added as a slurry in EtOH. After about 6 hours, the resulting slurry is cooled to about 20° C. over about 1 hour and is mixed at that temperature for about 36 hours. The slurry is filtered to recover a crystalline solid, which is an ethanol solvate of ABT-263 bis-HCl. Drying of this solid under vacuum and nitrogen with mild agitation for about 8 days yields white desolvated ABT-263 bis-HCl crystals. This material is suitable for preparation of an ABT-263 bis-HCl formulation of the present invention.

The term “free base” is used for convenience herein to refer to the parent compound, while recognizing that the parent compound is, strictly speaking, zwitterionic and thus does not always behave as a true base.

Compounds of Formula I, and methods of preparation of such compounds, are disclosed in above-cited U.S. Patent Application Publication No. 2007/0027135 and/or in above-cited U.S. Patent Application Publication No. 2007/0072860, each of which is incorporated herein by reference in its entirety. Terms for substituents used herein are defined exactly as in those publications.

Compounds of Formula I having —NH, —C(O)OH, —OH or —SH moieties may have attached thereto prodrug-forming moieties which can be removed by metabolic processes in vivo to release the parent compound having free —NH, —C(O)OH, —OH or —SH moieties. Salts of prodrugs can also be used.

Without being bound by theory, it is believed that the therapeutic efficacy of compounds of Formula I is due at least in part to their ability to bind to a Bcl-2 family protein such as Bcl-2, Bcl-X_{L} or Bcl-w in a way that inhibits the anti-apoptotic action of the protein, for example by occupying the BH3 binding groove of the protein. It will generally be found desirable to select a compound having high binding affinity for a Bcl-2 family protein, for example a Kᵢ not greater than about 5 nM, preferably not greater than about 1 nM.

A composition as provided herein comprising any specific compound disclosed in the '135 publication is expressly contemplated as an embodiment of the present invention.

In a more particular embodiment, the composition comprises N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino-3-((trifluoromethyl)sulfonyl)benzenesulfonamide (ABT-263) or a salt, prodrug, salt of a prodrug or metabolite thereof. In a still more particular embodiment, the composition comprises ABT-263 parent compound (i.e., free base) or a salt, prodrug or salt of a prodrug thereof. In a still more particular embodiment, the composition comprises ABT-263 free base or a salt thereof. In an even more particular embodiment, the composition comprises ABT-263 free base or ABT-263 bis-HCl.

The drug (i.e., a compound of Formula I or a salt, prodrug, salt of a prodrug or metabolite thereof) is present in the composition in an amount that can be therapeutically effective when the composition is administered to a subject in need thereof according to an appropriate regimen. Dosage amounts are expressed herein as parent-compound-equivalent amounts unless the context requires otherwise. Typically, a unit dose (the amount administered at a single time), which can be administered at an appropriate frequency, e.g., twice daily to once weekly, is about 10 to about 1,000 mg, depending on the compound in question. Where frequency of administration is once daily (q.d.), unit dose and daily dose are the same. Illustratively, for example where the drug is ABT-263, the unit dose is typically about 25 to about 1,000 mg, more typically about 50 to about 500 mg, for example about 50, about 100, about 150, about 200, about 250, about 300, about 350, about 400, about 450 or about 500 mg. Where the composition comprises a capsule shell enclosing the drug-carrier system, a unit dose can be deliverable in a single capsule or a plurality of capsules, most typically 1 to about 10 capsules.

The higher the unit dose, the more desirable it becomes to select a carrier that permits a relatively high concentration of the drug in solution therein. Typically, the concentration of drug in the drug-carrier system is at least about 10 mg/ml, e.g., about 10 to about 500 mg/ml, but lower and higher concentrations can be acceptable or achievable in specific cases. Illustratively, for example where the drug is ABT-263, the drug concentration in various embodiments is at least about 10 mg/ml, e.g., about 10 to about 400 mg/ml, or at least about 20 mg/ml, e.g., about 20 to about 200 mg/ml, for example about 20, about 25, about 30, about 40, about 50, about 75, about 100, about 125, about 150 or about 200 mg/ml.

In a composition of the invention, the drug is “in solution” in the carrier. This will be understood to mean that substantially all of the drug is in solution, i.e., no substantial portion, for example no more than about 2%, or no more than about 1%, of the drug is in solid (e.g., crystalline) form, whether dispersed, for example in the form of a suspension, or not. In practical terms, this means that the drug must normally be formulated at a concentration below its limit of solubility in the carrier. It will be understood that the limit of solubility can be temperature-dependent, thus selection of a suitable concentration should take into account the range of temperatures to which the composition is likely to be exposed in normal storage, transport and use.

The carrier is “substantially non-aqueous”, i.e., having no water, or having an amount of water that is small enough to be, in practical terms, essentially non-deleterious to performance or properties of the composition. Typically, the carrier comprises zero to less than about 5% by weight water. It will be understood that certain ingredients useful herein can bind small amounts of water on or within their molecules or supramolecular structures; such bound water if present does not affect the “substantially non-aqueous” character of the carrier as defined herein.

As indicated above, the carrier comprises two essential components: a phospholipid, and a pharmaceutically acceptable solubilizing agent for the phospholipid. Ethanol can optionally be present, for example as a component of the solubilizing agent, but if present is in an amount not greater than about 25% by weight of the carrier. It will be understood that reference in the singular to a (or the) phospholipid, solubilizing agent or other formulation ingredient herein includes the plural; thus combinations, for example mixtures, of more than one phospholipid, or more than one solubilizing agent, are expressly contemplated herein. The solubilizing agent, or the combination of solubilizing agent and phospholipid, also solubilizes the drug, although other carrier ingredients, such as a surfactant or an alcohol such as ethanol, optionally present in the carrier can in some circumstances provide enhanced solubilization of the drug.

Any pharmaceutically acceptable phospholipid or mixture of phospholipids can be used. In general such phospholipids are phosphoric acid esters that yield on hydrolysis phosphoric acid, fatty acid(s), an alcohol and a nitrogenous base. Pharmaceutically acceptable phospholipids can include without limitation phosphatidylcholines, phosphatidylserines and phosphatidylethanolamines. In one embodiment the composition comprises phosphatidylcholine, derived for example from natural lecithin. Any source of lecithin can be used, including animal sources such as egg yolk, but plant sources are generally preferred. Soy is a particularly rich source of lecithin that can provide phosphatidylcholine for use in the present invention.

Illustratively, a suitable amount of phospholipid is about 15% to about 75%, for example about 30% to about 60%, by weight of the carrier, although greater and lesser amounts can be useful in particular situations.

Ingredients useful as components of the solubilizing agent are not particularly limited and will depend to some extent on the particular drug and antioxidant and the desired concentration of each and of phospholipid. In one embodiment, the solubilizing agent comprises one or more glycols, one or more glycolides and/or one or more glyceride materials.

Suitable glycols include propylene glycol and polyethylene glycols (PEGs) having molecular weight of about 200 to about 1,000 g/mol, e.g., PEG-400, which has an average molecular weight of about 400 g/mol. Such glycols can provide relatively high solubility of the drug; however in some cases the drug, particularly a drug having a tendency for hydrolytic, solvolytic or oxidative instability, can exhibit chemical degradation to some degree when in solution in a carrier comprising such glycols. This can be evident by color changes of the drug solution with time. The higher the glycol content of the carrier, the greater may be the tendency for degradation of a chemically unstable drug. In one embodiment, therefore, one or more glycols are present in a total glycol amount of at least about 1% but less than about 50%, for example less than about 30%, less than about 20%, less than about 15% or less than about 10% by weight of the carrier. In another embodiment, the carrier comprises substantially no glycol.

Glycolides are glycols such as propylene glycol or PEG esterified with one or more organic acids, for example medium- to long-chain fatty acids. Suitable examples include propylene glycol monocaprylate, propylene glycol monolaurate and propylene glycol dilaurate products such as, for example. Capmul PG-8™, Capmul PG-12™ and Capmul PG-2L™ respectively of Abitec Corp. and products substantially equivalent thereto.

Suitable glyceride materials include, without limitation, medium to long chain mono-, di- and triglycerides. The term “medium chain” herein refers to hydrocarbyl chains individually having no less than about 6 and less than about 12 carbon atoms, including for example C₈ to C₁₀ chains. Thus glyceride materials comprising caprylyl and capryl chains, e.g., caprylic/capric mono-, di- and/or triglycerides, are examples of “medium chain” glyceride materials herein. The term “long chain” herein refers to hydrocarbyl chains individually having at least about 12, for example about 12 to about 18, carbon atoms, including for example lauryl, myristyl, cetyl, stearyl, oleyl, linoleyl and linolenyl chains. Medium to long chain hydrocarbyl groups in the glyceride materials can be saturated, mono- or polyunsaturated.

In one embodiment the carrier comprises, as a major component of the solubilizing agent, a medium chain and/or a long chain triglyceride material. A suitable example of a medium chain triglyceride material is a caprylic/capric triglyceride product such as, for example, Captex 355 EP™ of Abitec Corp. and products substantially equivalent thereto. Suitable examples of long chain triglycerides include any pharmaceutically acceptable vegetable oil, for example canola, coconut, corn, cottonseed, flaxseed, olive, palm, peanut, safflower, sesame, soy and sunflower oils, and mixtures of such oils. Oils of animal, particularly marine animal, origin can also be used, including for example fish oil.

Where one or more glyceride materials are present as a major component of the solubilizing agent, a suitable total amount of glycerides is an amount effective to solubilize the phospholipid and, in combination with other components of the carrier, effective to maintain the drug in solution. For example, glyceride materials such as medium chain and/or long chain triglycerides can be present in a total glyceride amount of about 5% to about 70%, for example about 15% to about 60% or about 25% to about 50%, by weight of the carrier, although greater and lesser amounts can be useful in particular situations. In one embodiment, the encapsulated liquid comprises about 7% to about 30%, for example about 10% to about 25%, by weight medium-chain triglycerides and about 7% to about 30%, for example about 10% to about 25%, by weight medium-chain mono- and diglycerides.

Additional solubilizing agents that are other than glycols or glyceride materials can be included if desired. Such agents, for example N-substituted amide solvents such as dimethylformamide (DMF) and N,N-dimethylacetamide (DMA), can, in specific cases, assist in raising the limit of solubility of the drug in the carrier, thereby permitting increased drug loading. However, the carriers useful herein generally provide adequate solubility of small-molecule drugs of interest herein without such additional agents.

Even when a sufficient amount of a glycol, glycolide or glyceride material is present to solubilize the phospholipid, the resulting carrier solution and/or the drug-carrier system may be rather viscous and difficult or inconvenient to handle. In such cases it may be found desirable to include in the carrier a viscosity reducing agent in an amount effective to provide acceptably low viscosity. An example of such an agent is an alcohol, more particularly ethanol, which is preferably introduced in a form that is substantially free of water, for example 99% ethanol, dehydrated alcohol USP or absolute ethanol. Excessively high concentrations of ethanol should, however, generally be avoided. This is particularly true where, for example, the drug-carrier system is to be administered in a gelatin capsule, because of the tendency of high ethanol concentrations to result in mechanical failure of the capsule. In general, suitable amounts of ethanol are 0% to about 25%, for example about 1% to about 20% or about 3% to about 15%, by weight of the carrier. Glycols such as propylene glycol or PEG and medium-chain mono- and diglycerides (for example caprylic/capric mono- and diglycerides) can also be helpful to lower viscosity; where the drug-carrier system is to be encapsulated in a hard capsule such as a hard gelatin capsule, medium-chain mono- and diglycerides are particularly useful in this regard.

Optionally, the carrier further comprises a pharmaceutically acceptable non-phospholipid surfactant. One of skill in the art will be able to select a suitable surfactant for use in a composition of the invention, based on information herein. Such a surfactant can serve various functions, including for example enhancing dispersion of the encapsulated liquid upon release from the capsule in the aqueous environment of the gastrointestinal tract. Thus in one embodiment the non-phospholipid surfactant is a dispersing and/or emulsifying agent that enhances dispersion and/or emulsification of the capsule contents in real or simulated gastrointestinal fluid. Illustratively, a surfactant such as a polysorbate (polyoxyethylene sorbitan ester), e.g., polysorbate 80 (available for example as Tween 80™ from Uniqema), can be included in an amount of 0% to about 30%, for example about 7% to about 30% or about 10% to about 25%, by weight of the carrier. In some embodiments such a surfactant is included in an amount of 0% to about 5%, for example 0% to about 2% or 0% to about 1%, by weight of the carrier.

Other ingredients can optionally be present in the carrier, selected for example from conventional formulation ingredients such as antioxidants, preservatives, colorants, flavorants and combinations thereof. As indicated above, the carrier can optionally comprise a solid or semi-solid substrate having the drug solution adsorbed therein or thereon. Examples of such substrates include particulate diluents such as lactose, starches, silicon dioxide, etc., and polymers such as polyacrylates, high molecular weight PEGs, or cellulose derivatives, e.g., hydroxypropylmethylcellulose (HPMC). Where a solid solution is desired, a high melting point ingredient such as a wax can be included. A solid drug-carrier system can optionally be encapsulated or, if desired, delivered in tablet form. The drug-carrier system can, in some embodiments, be adsorbed on, or impregnated into, a drug delivery device.

Conveniently, pre-blended products are available containing a suitable phospholipid+solubilizing agent combination for use in compositions of the present invention. It is emphasized that, while compositions comprising such products are embraced by the present invention, no limitation to such compositions is intended. Pre-blended phospholipid+solubilizing agent products can be advantageous in improving ease of preparation of the present compositions.

An illustrative example of a pre-blended phospholipid+solubilizing agent product is Phosal 50 PG™, available from Phospholipid GmbH, Germany, which comprises, by weight, not less than 50% phosphatidylcholine, not more than 6% lysophosphatidylcholine, about 35% propylene glycol, about 3% mono- and diglycerides from sunflower oil, about 2% soy fatty acids, about 2% ethanol, and about 0.2% ascorbyl palmitate.

Another illustrative example is Phosal 53 MCT™, also available from Phospholipid GmbH, which contains, by weight, not less than 53% phosphatidylcholine, not more than 6% lysophosphatidylcholine, about 29% medium chain triglycerides, 3-6% (typically about 5%) ethanol, about 3% mono- and diglycerides from sunflower oil, about 2% oleic acid, and about 0.2% ascorbyl palmitate (reference composition). A product having the above or substantially equivalent composition, whether sold under the Phosal 53 MCT™brand or otherwise, is generically referred to herein as “phosphatidylcholine+medium chain triglycerides 53/29”. A product having “substantially equivalent composition” in the present context means having a composition sufficiently similar to the reference composition in its ingredient list and relative amounts of ingredients to exhibit no practical difference in properties with respect to utilization of the product herein.

Yet another illustrative example is Lipoid S75™, available from Lipoid GmbH, which contains, by weight, not less than 70% phosphatidylcholine in a solubilizing system. This can be further blended with medium-chain triglycerides, for example in a 30/70 weight/weight mixture, to provide a product (“Lipoid S75™ MCT”) containing, by weight, not less than 20% phosphatidylcholine, 2-4% phosphatidylethanolamine, not more than 1.5% lysophosphatidylcholine, and 67-73% medium-chain triglycerides.

Yet another illustrative example is Phosal 50 SA+™, also available from Phospholipid GmbH, which contains, by weight, not less than 50% phosphatidylcholine and not more than 6% lysophosphatidylcholine in a solubilizing system comprising safflower oil and other ingredients.

The phosphatidylcholine component of each of these pre-blended products is derived from soy lecithin. Products of substantially equivalent composition may be obtainable from other suppliers.

A pre-blended product such as Phosal 50 PG™, Phosal 53 MCT™, Lipoid S75™ MCT or Phosal 50 SA+™can, in some embodiments, constitute substantially the entire carrier system (other than the antioxidant as provided herein). In other embodiments, additional ingredients are present, for example medium-chain mono- and/or diglycerides, ethanol (additional to any that may be present in the pre-blended product), a non-phospholipid surfactant such as polysorbate 80, polyethylene glycol and/or other ingredients. Such additional ingredients, if present, are typically included in only minor amounts. Illustratively, phosphatidylcholine+medium chain triglycerides 53/29 can be included in the carrier in an amount of about 50% to 100%, for example about 80% to 100%, by weight of the carrier.

In some embodiments of the invention, the drug-carrier system is dispersible in an aqueous phase to form a non-gelling, substantially non-transparent liquid dispersion. This property can readily be tested by one of skill in the art, for example by adding 1 part of the drug-carrier system to about 20 parts of water with agitation at ambient temperature and assessing gelling behavior and transparency of the resulting dispersion. Compositions having ingredients in relative amounts as indicated herein will generally be found to pass such a test, i.e., to form a liquid dispersion that does not gel and is substantially non-transparent. In “non-gelling” embodiments, the composition does not contain a gel-promoting agent in a gel-promoting effective amount. If gelling behavior is desired, such an agent can be added. A “substantially non-transparent” dispersion is believed to be formed on mixing with an aqueous phase a composition of the invention having any substantial amount of the phospholipid component. However, for clarification it is emphasized that compositions of the invention themselves, being substantially non-aqueous, are generally clear and transparent. In this regard, it is noted that phospholipids tend to form bi- and multilamellar aggregates when placed in an aqueous environment, such aggregates generally being large enough to scatter transmitted light and thereby provide a non-transparent, e.g., cloudy, dispersion. In the case of phosphatidylcholine+medium chain triglycerides 53/29, for example, dispersion in an aqueous environment typically forms not only multilamellar aggregates but also a coarse oil-in-water emulsion. Presence of multilamellar aggregates can often be confirmed by microscopic examination in presence of polarized light, such aggregates tending to exhibit birefringence, for example generating a characteristic “Maltese cross” pattern.

Without being bound by theory, it is believed that behavior of the drug-carrier system of a composition of the invention upon mixing with an aqueous phase is indicative of how the composition interacts with gastrointestinal fluid following oral administration to a subject. Although formation of a gel can be useful for controlled-release topical delivery of a drug, it is believed that gelling would be detrimental to efficient gastrointestinal absorption. For this reason, embodiments of the invention described above, wherein the drug-carrier system does not gel when mixed with an aqueous phase, are generally preferred. It is further believed, again without being bound by theory, that formation of bi- and multilamellar aggregates in the gastrointestinal fluid, as evidenced by non-transparency of the dispersion formed upon mixing the drug-carrier system with an aqueous phase, can be an important factor in providing the relatively high bioavailability of certain compositions of the invention when administered orally.

Illustratively where the drug is ABT-263, the carrier ingredients and amounts thereof are selected to provide solubility of the drug in the carrier of at least about 10 mg/ml, for example at least about 20 mg/ml, at about 25° C.

A particular composition of the present invention, referred to herein as “Formulation C”, consists of ABT-263 bis-HCl in solution at a free base equivalent concentration of 25 mg/ml in a carrier liquid consisting of 90% phosphatidylcholine+medium chain triglycerides 53/29 and 10% dehydrated alcohol USP (meeting standards set forth in the United States Pharmacopeia).

In certain embodiments, the carrier ingredients and amounts thereof are selected to provide enhanced bioabsorption by comparison with a standard solution of the drug, e.g., a solution in a carrier consisting of 10% DMSO in PEG-400, when administered orally. Such enhanced bioabsorption can be evidenced, for example, by a pharmacokinetic (PK) profile having one or more of a higher Cₘₐₓ or an increased bioavailability as measured by AUC, for example AUC₀₋₂₄ or AUC_{0-∞}. Illustratively, bioavailability can be expressed as a percentage, for example using the parameter F, which computes AUC for oral delivery of a test composition as a percentage of AUC for intravenous (i.v.) delivery of the drug in a suitable solvent, taking into account any difference between oral and i.v. doses.

Bioavailability can be determined by PK studies in humans or in any suitable model species. For present purposes, a dog model, as illustratively described in Example 3 below, is generally suitable. In various illustrative embodiments, where the drug is ABT-263, compositions of the invention exhibit oral bioavailability of at least about 30%, at least about 35% or at least about 40%, up to or exceeding about 50%, in a dog model, when administered as a single dose of about 2.5 to about 10 mg/kg to fasting or non-fasting animals.

In one example, the composition comprises ABT-263 and a carrier comprising ingredients and amounts thereof selected to provide (a) solubility of ABT-263 of at least about 20 mg/ml at about 25° C.; and (b) a PK profile upon oral administration of the composition in a dog model exhibiting a bioavailability of at least about 30%.

In another example, the composition comprises ABT-263 and a carrier comprising ingredients and amounts thereof selected to provide (a) solubility of ABT-263 of at least about 25 mg/ml at about 25° C.; and (b) a PK profile upon oral administration of the composition in a dog model exhibiting a bioavailability of at least about 40%.

The potential of the present invention to provide bioavailability, for example of ABT-263, substantially greater, for example at least about 1.5× or at least about 2× greater, than that of the solution in 10% DMSO in PEG-400 described in above-cited U.S. Patent Application Publication No. 2007/0027135, is an unexpected benefit of great practical value, especially in view of the fact that formulation changes apparently have little effect on bioavailability of earlier generations of Bcl-2 protein family inhibitors such as ABT-737. As illustratively described in Example 3 below, bioavailability in a rat model of ABT-737, formulated in 90% phosphatidylcholine+medium chain triglycerides 53/29 and 10% ethanol, was only 3.3%, not markedly different from that of other formulations tested.

The present invention is not limited by the process used to prepare a composition as embraced or described herein. Any suitable process of pharmacy can be used. Illustratively, compositions of the invention can be prepared by a process comprising simple mixing of the recited ingredients, wherein order of addition is not critical, to form a drug-carrier system. It is noted, however, that if the phospholipid component is used in its solid state, for example in the form of soy lecithin, it will generally be desirable to first solubilize the phospholipid with the solubilizing agent component or part thereof. Thereafter other ingredients of the carrier, if any, and the drug can be added by simple mixing, with agitation as appropriate. As mentioned above, use of a pre-blended product comprising phospholipid and solubilizing agent can simplify preparation of the composition. An illustrative process employing such a product, in this case phosphatidylcholine+medium chain triglycerides 53/29, is presented in Example 1 below. Optionally, the drug-carrier system can be used as a premix for capsule filling, as illustrated in Example 2 below. The term “filling” used in relation to a capsule herein means placement of a desired amount of a composition in a capsule shell, and should not be taken to mean that all space in the capsule is necessarily occupied by the composition.

Compositions embraced herein, including compositions described generally or with specificity herein, are useful for orally delivering a drug that is a compound of Formula I or a pharmaceutically acceptable salt, prodrug, salt of a prodrug or metabolite thereof to a subject. Accordingly, a method of the invention for delivering such a drug to a subject comprises orally administering a composition as described above.

The subject can be human or non-human (e.g., a farm, zoo, work or companion animal, or a laboratory animal used as a model) but in an important embodiment the subject is a human patient in need of the drug, for example to treat a disease characterized by apoptotic dysfunction and/or overexpression of an anti-apoptotic Bcl-2 family protein. A human subject can be male or female and of any age. The patient is typically an adult, but a method of the invention can be useful to treat a childhood cancer such as leukemia, for example acute lymphocytic leukemia, in a pediatric patient.

The composition is normally administered in an amount providing a therapeutically effective daily dose of the drug. The term “daily dose” herein means the amount of drug administered per day, regardless of the frequency of administration. For example, if the subject receives a unit dose of 150 mg twice daily, the daily dose is 300 mg. Use of the term “daily dose” will be understood not to imply that the specified dosage amount is necessarily administered once daily. However, in a particular embodiment the dosing frequency is once daily (q.d.), and the daily dose and unit dose are in this embodiment the same thing.

What constitutes a therapeutically effective dose depends on the particular compound, the subject (including species and body weight of the subject), the disease (e.g., the particular type of cancer) to be treated, the stage and/or severity of the disease, the individual subject's tolerance of the compound, whether the compound is administered in monotherapy or in combination with one or more other drugs, e.g., other chemotherapeutics for treatment of cancer, and other factors. Thus the daily dose can vary within wide margins, for example from about 10 to about 1,000 mg. Greater or lesser daily doses can be appropriate in specific situations. It will be understood that recitation herein of a “therapeutically effective” dose herein does not necessarily require that the drug be therapeutically effective if only a single such dose is administered; typically therapeutic efficacy depends on the composition being administered repeatedly according to a regimen involving appropriate frequency and duration of administration. It is strongly preferred that, while the daily dose selected is sufficient to provide benefit in terms of treating the cancer, it should not be sufficient to provoke an adverse side-effect to an unacceptable or intolerable degree. A suitable therapeutically effective dose can be selected by the physician of ordinary skill without undue experimentation based on the disclosure herein and on art cited herein, taking into account factors such as those mentioned above. The physician may, for example, start a cancer patient on a course of therapy with a relatively low daily dose and titrate the dose upwards over a period of days or weeks, to reduce risk of adverse side-effects.

Illustratively, suitable doses of ABT-263 are generally about 25 to about 1,000 mg/day, more typically about 50 to about 500 mg/day or about 200 to about 400 mg/day, for example about 50, about 100, about 150, about 200, about 250, about 300, about 350, about 400, about 450 or about 500 mg/day, administered at an average dosage interval of about 3 hours to about 7 days, for example about 8 hours to about 3 days, or about 12 hours to about 2 days. In most cases a once-daily (q.d.) administration regimen is suitable.

An “average dosage interval” herein is defined as a span of time, for example one day or one week, divided by the number of unit doses administered over that span of time. For example, where a drug is administered three times a day, around 8 am, around noon and around 6 pm, the average dosage interval is 8 hours (a 24-hour time span divided by 3). If the drug is formulated as a discrete dosage form such as a tablet or capsule, a plurality (e.g., 2 to about 10) of dosage forms administered at one time is considered a unit dose for the purpose of defining the average dosage interval.

Where the drug compound is ABT-263, for example in the form of ABT-263 free base or ABT-263 bis-HCl, a daily dosage amount and dosage interval can, in some embodiments, be selected to maintain a plasma concentration of ABT-263 in a range of about 0.5 to about 10 μg/ml. Thus, during a course of ABT-263 therapy according to such embodiments, the steady-state peak plasma concentration (Cₘₐₓ) should in general not exceed about 10 μg/ml, and the steady-state trough plasma concentration (Cₘᵢₙ) should in general not fall below about 0.5 μg/ml. It will further be found desirable to select, within the ranges provided above, a daily dosage amount and average dosage interval effective to provide a Cₘₐₓ/Cₘᵢₙ ratio not greater than about 5, for example not greater than about 3, at steady-state. It will be understood that longer dosage intervals will tend to result in greater Cₘₐₓ/Cₘᵢₙ ratios. Illustratively, at steady-state, an ABT-263 Cₘₐₓ of about 3 to about 8 μg/ml and Cₘᵢₙ of about 1 to about 5 μg/ml can be targeted by the present method. Steady-state values of Cₘₐₓ and Cₘᵢₙ can be established in a human PK study, for example conducted according to standard protocols including but not limited to those acceptable to a regulatory agency such as the U.S. Food and Drug Administration (FDA).

Where the composition is in the form of an unencapsulated liquid, the composition can be swallowed neat, but administration is generally more convenient and pleasant if the composition is first diluted in a suitable imbibable liquid. Suitable liquid diluents include without limitation any aqueous beverage such as water, milk, fruit juice (e.g., apple juice, grape juice, orange juice, etc.), carbonated drink, enteral nutrition formula, energy drink, tea or coffee. Where a liquid diluent is to be used, the composition should be mixed with the diluent using sufficient agitation (e.g., by shaking and/or stiffing) to thoroughly disperse the composition in the diluent, and administered immediately thereafter, so that the composition does not separate from the diluent before swallowing. If desired the diluent can be in the form of a part-frozen slurry such as a slush or smoothie. Any convenient rate of dilution can be employed, for example about 1 to about 100, or about 5 to about 50, parts by volume of the composition per part by volume of the diluent.

Where the composition is in the form of a capsule, one to a small plurality of capsules can be swallowed whole, typically with the aid of water or other imbibable liquid to help the swallowing process. Suitable capsule shell materials include, without limitation, gelatin (in the form of hard gelatin capsules or soft elastic gelatin capsules), starch, carrageenan and HPMC. Where the drug-carrier system is liquid, soft elastic gelatin capsules are generally preferred.

For administering ABT-263 according to the present method, the drug is illustratively present in the pharmaceutical composition in the form of ABT-263 free base or ABT-263 bis-HCl. Any ABT-263 composition of the present invention, as defined more fully above, can be used. In one aspect of the present method, the composition administered is Formulation C as described above or a composition of the present invention that is substantially bioequivalent to Formulation C.

The term “substantially bioequivalent” herein means exhibiting, in a human PK single- or multiple-dose study in fasting or non-fasting conditions, substantially equal peak plasma concentration (Cₘₐₓ) and substantially equal exposure measured as area under the plasma concentration-time curve, calculated from zero to 24 hours from time of administration (AUC₀₋₂₄) or from zero to infinity (AUC_{0-∞}). The compositions being compared for substantial bioequivalence should be administered at the same dose or doses, expressed in the case of ABT-263 as free base equivalent. If a multiple-dose study is used to draw the comparison, it is the steady-state values of Cₘₐₓ and AUC that are used. In the present context, Cₘₐₓ or AUC of a test composition is “substantially equal” if it is no less than 80% and no greater than 125% of the corresponding parameter in a reference composition (e.g., Formulation C as described above).

As compositions of the present invention typically exhibit only a minor food effect, administration according to the present embodiment can be with or without food, i.e., in a non-fasting or fasting condition. It is generally preferred to administer the present compositions to a non-fasting patient.

Compositions of the invention are suitable for use in monotherapy or in combination therapy, for example with other chemotherapeutics or with ionizing radiation. A particular advantage of the present invention is that it permits once-daily oral administration, a regimen which is convenient for the patient who is undergoing treatment with other orally administered drugs on a once-daily regimen. Oral administration is easily accomplished by the patient him/herself or by a caregiver in the patient's home; it is also a convenient route of administration for patients in a hospital or residential care setting.

Combination therapies illustratively include administration of a composition of the present invention, for example such a composition comprising ABT-263, concomitantly with one or more of bortezomid, carboplatin, cisplatin, cyclophosphamide, dacarbazine, dexamethasone, docetaxel, doxorubicin, etoposide, fludarabine, hydroxydoxorubicin, irinotecan, paclitaxel, rapamycin, rituximab, vincristine and the like, for example with a polytherapy such as CHOP (cyclophosphamide+hydroxydoxorubicin+vincristine+prednisone), RCVP (rituximab+cyclophosphamide+vincristine+prednisone), R-CHOP (rituximab+CHOP) or DA-EPOCH-R (dose-adjusted etoposide, prednisone, vincristine, cyclophosphamide, doxorubicin and rituximab).

A composition of the invention, for example such a composition comprising ABT-263, can be administered in combination therapy with one or more therapeutic agents that include, but are not limited to, angiogenesis inhibitors, antiproliferative agents, other apoptosis promoters (for example, Bcl-xL, Bcl-w and Bfl-1 inhibitors), activators of a death receptor pathway, BiTE (bi-specific T-cell engager) antibodies, dual variable domain binding proteins (DVDs), inhibitors of apoptosis proteins (IAPs), microRNAs, mitogen-activated extracellular signal-regulated kinase inhibitors, multivalent binding proteins, poly-ADP (adenosine diphosphate)-ribose polymerase (PARP) inhibitors, small inhibitory ribonucleic acids (siRNAs), kinase inhibitors, receptor tyrosine kinase inhibitors, aurora kinase inhibitors, polo-like kinase inhibitors, bcr-abl kinase inhibitors, growth factor inhibitors, COX-2 inhibitors, non-steroidal anti-inflammatory drugs (NSAIDs), antimitotic agents, alkylating agents, antimetabolites, intercalating antibiotics, platinum-containing chemotherapeutic agents, growth factor inhibitors, ionizing radiation, cell cycle inhibitors, enzymes, topoisomerase inhibitors, biologic response modifiers, immunologicals, antibodies, hormonal therapies, retinoids, deltoids, plant alkaloids, proteasome inhibitors, HSP-90 inhibitors, histone deacetylase (HDAC) inhibitors, purine analogs, pyrimidine analogs, MEK inhibitors, CDK inhibitors, ErbB2 receptor inhibitors, mTOR inhibitors as well as other antitumor agents.

Angiogenesis inhibitors include, but are not limited to, EGFR inhibitors, PDGFR inhibitors, VEGFR inhibitors, TIE2 inhibitors, IGF1R inhibitors, matrix metalloproteinase 2 (MMP-2) inhibitors, matrix metalloproteinase 9 (MMP-9) inhibitors and thrombospondin analogs.

Examples of EGFR inhibitors include, but are not limited to, gefitinib, erlotinib, cetuximab, EMD-7200, ABX-EGF, HR3, IgA antibodies, TP-38 (IVAX), EGFR fusion protein, EGF-vaccine, anti-EGFR immunoliposomes and lapatinib.

Examples of PDGFR inhibitors include, but are not limited to, CP-673451 and CP-868596.

Examples of VEGFR inhibitors include, but are not limited to, bevacizumab, sunitinib, sorafenib, CP-547632, axitinib, vandetanib, AEE788, AZD-2171, VEGF trap, vatalanib, pegaptanib, IM862, pazopanib, ABT-869 and angiozyme.

Bcl-2 family protein inhibitors other than ABT-263 or compounds of Formula I herein include, but are not limited to, AT-101 ((−)gossypol), Genasense™ Bcl-2-targeting antisense oligonucleotide (G3139 or oblimersen), IPI-194, IPI-565, ABT-737, GX-070 (obatoclax) and the like.

Activators of a death receptor pathway include, but are not limited to, TRAIL, antibodies or other agents that target death receptors (e.g., DR4 and DR5) such as apomab, conatumumab, ETR2-ST01, GDC0145 (lexatumumab), HGS-1029, LBY-135, PRO-1762 and trastuzumab.

Examples of thrombospondin analogs include, but are not limited to, TSP-1, ABT-510, ABT-567 and ABT-898.

Examples of aurora kinase inhibitors include, but are not limited to, VX-680, AZD-1152 and MLN-8054.

An example of a polo-like kinase inhibitor includes, but is not limited to, BI-2536.

Examples of bcr-abl kinase inhibitors include, but are not limited to, imatinib and dasatinib.

Examples of platinum-containing agents include, but are not limited to, cisplatin, carboplatin, eptaplatin, lobaplatin, nedaplatin, oxaliplatin and satraplatin.

Examples of mTOR inhibitors include, but are not limited to, CCI-779, rapamycin, temsirolimus, everolimus, RAD001 and AP-23573.

Examples of HSP-90 inhibitors include, but are not limited to, geldanamycin, radicicol, 17-AAG, KOS-953, 17-DMAG, CNF-101, CNF-1010, 17-AAG-nab, NCS-683664, efungumab, CNF-2024, PU3, PU24FCl, VER-49009, IPI-504, SNX-2112 and STA-9090.

Examples of HDAC inhibitors include, but are not limited to, suberoylanilide hydroxamic acid (SAHA), MS-275, valproic acid, TSA, LAQ-824, trapoxin and depsipeptide.

Examples of MEK inhibitors include, but are not limited to, PD-325901, ARRY-142886, ARRY-438162 and PD-98059.

Examples of CDK inhibitors include, but are not limited to, flavopyridol, MCS-5A, CVT-2584, seliciclib ZK-304709, PHA-690509, BMI-1040, GPC-286199, BMS-387032, PD-332991 and AZD-5438.

Examples of COX-2 inhibitors include, but are not limited to, celecoxib, parecoxib, deracoxib, ABT-963, etoricoxib, lumiracoxib, BMS-347070, RS 57067, NS-398, valdecoxib, rofecoxib, SD-8381, 4-methyl-2-(3,4-dimethylphenyl)-1-(4-sulfamoylphenyl)-1H-pyrrole, T-614, JTE-522, S-2474, SVT-2016, CT-3 and SC-58125.

Examples of NSAIDs include, but are not limited to, salsalate, diflunisal, ibuprofen, ketoprofen, nabumetone, piroxicam, naproxen, diclofenac, indomethacin, sulindac, tolmetin, etodolac, ketorolac and oxaprozin.

Examples of ErbB2 receptor inhibitors include, but are not limited to, CP-724714, canertinib, trastuzumab, petuzumab, TAK-165, ionafamib, GW-282974, EKB-569, PI-166, dHER2, APC-8024, anti-HER/2neu bispecific antibody B7.her2IgG3 and HER2 trifunctional bispecific antibodies mAB AR-209 and mAB 2B-1.

Examples of alkylating agents include, but are not limited to, nitrogen mustard N-oxide, cyclophosphamide, ifosfamide, trofosfamide, chlorambucil, melphalan, busulfan, mitobronitol, carboquone, thiotepa, ranimustine, nimustine, Cloretazine™ (laromustine), AMD-473, altretamine, AP-5280, apaziquone, brostallicin, bendamustine, carmustine, estramustine, fotemustine, glufosfamide, KW-2170, mafosfamide, mitolactol, lomustine, treosulfan, dacarbazine and temozolomide.

Examples of antimetabolites include, but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, pemetrexed, gemcitabine, fludarabine, 5-azacitidine, capecitabine, cladribine, clofarabine, decitabine, eflornithine, ethenylcytidine, cytosine arabinoside, hydroxyurea, TS-1, melphalan, nelarabine, nolatrexed, disodium pemetrexed, pentostatin, pelitrexol, raltitrexed, triapine, trimetrexate, vidarabine, mycophenolic acid, ocfosfate, pentostatin, tiazofurin, ribavirin, EICAR, hydroxyurea and deferoxamine.

Examples of antibiotics include, but are not limited to, intercalating antibiotics, aclarubicin, actinomycin D, amrubicin, annamycin, adriamycin, bleomycin, daunorubicin, doxorubicin (including liposomal doxorubicin), elsamitrucin, epirubicin, glarubicin, idarubicin, mitomycin C, nemorubicin, neocarzinostatin, peplomycin, pirarubicin, rebeccamycin, stimalamer, streptozocin, valrubicin, zinostatin and combinations thereof.

Examples of topoisomerase inhibiting agents include, but are not limited to, aclarubicin, amonafide, belotecan, camptothecin, 10-hydroxycamptothecin, 9-amino-camptothecin, amsacrine, dexrazoxane, diflomotecan, irinotecan HCl, edotecarin, epirubicin, etoposide, exatecan, becatecarin, gimatecan, lurtotecan, orathecin, BN-80915, mitoxantrone, pirarbucin, pixantrone, rubitecan, sobuzoxane, SN-38, tafluposide and topotecan.

Examples of antibodies include, but are not limited to, rituximab, cetuximab, bevacizumab, trastuzumab, CD40-specific antibodies and IGF1R-specific antibodies, chTNT-1/B, denosumab, edrecolomab, WX G250, zanolimumab, lintuzumab and ticilimumab.

Examples of hormonal therapies include, but are not limited to, sevelamer carbonate, rilostane, luteinizing hormone releasing hormone, modrastane, exemestane, leuprolide acetate, buserelin, cetrorelix, deslorelin, histrelin, anastrozole, fosrelin, goserelin, degarelix, doxercalciferol, fadrozole, formestane, tamoxifen, arzoxifene, bicalutamide, abarelix, triptorelin, finasteride, fulvestrant, toremifene, raloxifene, trilostane, lasofoxifene, letrozole, flutamide, megesterol, mifepristone, nilutamide, dexamethasone, prednisone and other glucocorticoids.

Examples of retinoids or deltoids include, but are not limited to, seocalcitol, lexacalcitol, fenretinide, aliretinoin, tretinoin, bexarotene and LGD-1550.

Examples of plant alkaloids include, but are not limited to, vincristine, vinblastine, vindesine and vinorelbine.

Examples of proteasome inhibitors include, but are not limited to, bortezomib, MG-132, NPI-0052 and PR-171.

Examples of immunologicals include, but are not limited to, interferons and numerous other immune-enhancing agents. Interferons include interferon alpha, interferon alpha-2a, interferon alpha-2b, interferon beta, interferon gamma-1a, interferon gamma-1b, interferon gamma-n1 and combinations thereof. Other agents include filgrastim, lentinan, sizofilan, BCG live, ubenimex, WF-10 (tetrachlorodecaoxide or TCDO), aldesleukin, alemtuzumab, BAM-002, dacarbazine, daclizumab, denileukin, gemtuzumab ozogamicin, ibritumomab, imiquimod, lenograstim, melanoma vaccine, molgramostim, sargaramostim, tasonermin, tecleukin, thymalasin, tositumomab, Virulizin™ immunotherapeutic of Lorus Pharmaceuticals, Z-100 (specific substance of Maruyama or SSM), Zevalin™ (90Y-ibritumomab tiuxetan), epratuzumab, mitumomab, oregovomab, pemtumomab, Provenge™ (sipuleucel-T), teceleukin, Therocys™ (Bacillus Calmette-Guerin), cytotoxic lymphocyte antigen 4 (CTLA4) antibodies and agents capable of blocking CTLA4 such as MDX-010.

Examples of biological response modifiers are agents that modify defense mechanisms of living organisms or biological responses, such as survival, growth, or differentiation of tissue cells to direct them to have anti-tumor activity. Such agents include, but are not limited to, krestin, lentinan, sizofuran, picibanil, PF-3512676 and ubenimex.

Examples of pyrimidine analogs include, but are not limited to, 5-fluorouracil, floxuridine, doxifluridine, raltitrexed, cytarabine, cytosine arabinoside, fludarabine, triacetyluridine, troxacitabine and gemcitabine.

Examples of purine analogs include, but are not limited to, mercaptopurine and thioguanine.

Examples of antimitotic agents include, but are not limited to, N-(2-((4-hydroxyphenyl)amino)pyridin-3-yl)-4-methoxybenzenesulfonamide, paclitaxel, docetaxel, larotaxel, epothilone D, PNU-100940, batabulin, ixabepilone, patupilone, XRP-9881, vinflunine and ZK-EPO (synthetic epothilone).

Examples of radiotherapy include, but are not limited to, external beam radiotherapy (XBRT), teletherapy, brachytherapy, sealed-source radiotherapy and unsealed-source radiotherapy.

BiTE antibodies are bi-specific antibodies that direct T-cells to attack cancer cells by simultaneously binding the two cells. The T-cell then attacks the target cancer cell. Examples of BiTE antibodies include, but are not limited to, adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (Sutton et al. (1997) J. Immunol. 158:5783-5790).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand. For example, siRNAs targeting Mcl-1 have been shown to enhance the activity of ABT-263 (Tse et al. (2008) Cancer Res. 68:3421-3428 and references therein).

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy-chain DVD polypeptides and two light-chain DVD polypeptides are referred to as DVD Ig's. Each half of a DVD Ig comprises a heavy-chain DVD polypeptide, a light-chain DVD polypeptide, and two antigen binding sites. Each binding site comprises a heavy-chain variable domain and a light-chain variable domain with a total of 6 CDRs involved in antigen binding per antigen binding site.

PARP inhibitors include, but are not limited to, ABT-888, olaparib, KU-59436, AZD-2281, AG-014699, BSI-201, BGP-15, INO-1001, ONO-2231 and the like.

Additionally or alternatively, a composition of the invention, for example such a composition comprising ABT-263, can be administered in combination therapy with one or more antitumor agents selected from ABT-100, N-acetylcolchinol-O-phosphate, acitretin, AE-941, aglycon protopanaxadiol, arglabin, arsenic trioxide, AS04 adjuvant-adsorbed HPV vaccine, L-asparaginase, atamestane, atrasentan, AVE-8062, bosentan, canfosfamide, Canvaxin™, catumaxomab, CeaVac™, celmoleukin, combrestatin A4P, contusugene ladenovec, Cotara™, cyproterone, deoxycoformycin, dexrazoxane, N,N-diethyl-2-(4-(phenylmethyl)phenoxy)ethanamine, 5,6-dimethylxanthenone-4-acetic acid, docosahexaenoic acid/paclitaxel, discodermolide, efaproxiral, enzastaurin, epothilone B, ethynyluracil, exisulind, falimarev, Gastrimmune™, GMK vaccine, GVAX™, halofuginone, histamine, hydroxycarbamide, ibandronic acid, ibritumomab tiuxetan, IL-13-PE38, inalimarev, interleukin 4, KSB-311, lanreotide, lenalidomide, lonafarnib, lovastatin, 5,10-methylenetetrahydrofolate, mifamurtide, miltefosine, motexafin, oblimersen, OncoVAX™ Osidem™, paclitaxel albumin-stabilized nanoparticle, paclitaxel poliglumex, pamidronate, panitumumab, peginterferon alfa, pegaspargase, phenoxodiol, poly(I)-poly(C12U), procarbazine, ranpirnase, rebimastat, recombinant quadrivalent HPV vaccine, squalamine, staurosporine, STn-KLH vaccine, T4 endonuclase V, tazarotene, 6,6′,7,12-tetramethoxy-2,2′-dimethyl-1β-berbaman, thalidomide, TNFerade™, ¹³¹I-tositumomab, trabectedin, triazone, tumor necrosis factor, Ukrain™, vaccinia-MUC-1 vaccine, L-valine-L-boroproline, Vitaxin™, vitespen, zoledronic acid and zorubicin.

In one embodiment, a composition of the invention, for example such a composition comprising ABT-263, is administered in a therapeutically effective amount to a subject in need thereof to treat a disease during which is overexpressed one or more of antiapoptotic Bcl-2 protein, antiapoptotic Bcl-X_{L} protein and antiapoptotic Bcl-w protein.

In another embodiment, a composition of the invention, for example such a composition comprising ABT-263, is administered in a therapeutically effective amount to a subject in need thereof to treat a disease of abnormal cell growth and/or dysregulated apoptosis.

Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In a more particular embodiment, a composition of the invention, for example such a composition comprising ABT-263, is administered in a therapeutically effective amount to a subject in need thereof to treat bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer or spleen cancer.

According to any of these embodiments, the composition can be administered in monotherapy or in combination therapy with one or more additional therapeutic agents.

For example, a method for treating mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof in a subject comprises administering to the subject therapeutically effective amounts of (a) a composition of the invention, for example such a composition comprising ABT-263, and (b) one or more of etoposide, vincristine, CHOP, rituximab, rapamycin, R-CHOP, RCVP, DA-EPOCH-R or bortezomib.

In particular embodiments, a composition of the invention, for example such a composition comprising ABT-263, is administered in a therapeutically effective amount to a subject in need thereof in monotherapy or in combination therapy with etoposide, vincristine, CHOP, rituximab, rapamycin, R-CHOP, RCVP, DA-EPOCH-R or bortezomib in a therapeutically effective amount, for treatment of a lymphoid malignancy such as B-cell lymphoma or non-Hodgkin's lymphoma.

In other particular embodiments, a composition of the invention, for example such a composition comprising ABT-263, is administered in a therapeutically effective amount to a subject in need thereof in monotherapy or in combination therapy with etoposide, vincristine, CHOP, rituximab, rapamycin, R-CHOP, RCVP, DA-EPOCH-R or bortezomib in a therapeutically effective amount, for treatment of chronic lymphocytic leukemia or acute lymphocytic leukemia.

The present invention also provides a method for maintaining in bloodstream of a human cancer patient a therapeutically effective plasma concentration of ABT-263 and/or one or more metabolites thereof, comprising administering to the subject a pharmaceutical composition comprising a drug-carrier system that comprises ABT-263 or a pharmaceutically acceptable salt, prodrug, salt of a prodrug or metabolite thereof, in solution in a substantially non-aqueous carrier that comprises a phospholipid component and a pharmaceutically acceptable solubilizing component, in a dosage amount equivalent to about 50 to about 500 mg ABT-263 per day, at an average dosage interval of about 3 hours to about 7 days.

What constitutes a therapeutically effective plasma concentration depends inter alfa on the particular cancer present in the patient, the stage, severity and aggressiveness of the cancer, and the outcome sought (e.g., stabilization, reduction in tumor growth, tumor shrinkage, reduced risk of metastasis, etc.). It is strongly preferred that, while the plasma concentration is sufficient to provide benefit in terms of treating the cancer, it should not be sufficient to provoke an adverse side-effect to an unacceptable or intolerable degree.

For treatment of cancer in general and of a lymphoid malignancy such as non-Hodgkin's lymphoma in particular, the plasma concentration of ABT-263 should in most cases be maintained in a range of about 0.5 to about 10 μg/ml. Thus, during a course of ABT-263 therapy, the steady-state Cₘₐₓ should in general not exceed about 10 μg/ml, and the steady-state Cₘᵢₙ should in general not fall below about 0.5 μg/ml. It will further be found desirable to select, within the ranges provided above, a daily dosage amount and average dosage interval effective to provide a Cₘₐₓ/Cₘᵢₙ ratio not greater than about 5, for example not greater than about 3, at steady-state. It will be understood that longer dosage intervals will tend to result in greater Cₘₐₓ/Cₘᵢₙ ratios. Illustratively, at steady-state, an ABT-263 Cₘₐₓ of about 3 to about 8 μg/ml and Cₘᵢₙ, of about 1 to about 5 μg/ml can be targeted by the present method.

A daily dosage amount effective to maintain a therapeutically effective ABT-263 plasma level is, according to the present embodiment, about 50 to about 500 mg. In most cases a suitable daily dosage amount is about 200 to about 400 mg. Illustratively, the daily dosage amount can be for example about 50, about 100, about 150, about 200, about 250, about 300, about 350, about 400, about 450 or about 500 mg.

An average dosage interval effective to maintain a therapeutically effective ABT-263 plasma level is, according to the present embodiment, about 3 hours to about 7 days. In most cases a suitable average dosage interval is about 8 hours to about 3 days, or about 12 hours to about 2 days. A once-daily (q.d.) administration regimen is often suitable.

For the present embodiment, ABT-263 is illustratively present in the pharmaceutical composition in the form of ABT-263 free base or ABT-263 bis-HCl. Any ABT-263 composition of the present invention, as defined more fully above, can be used. In one aspect of the present embodiment, the composition administered is (a) a prototype formulation consisting essentially of, or consisting of, a 25 mg/ml solution of ABT-263 bis-HCl in a carrier consisting of 90% by weight phospholipid/medium chain triglyceride 53/29 and 10% by weight dehydrated alcohol USP, or (b) a composition of the present invention that is substantially bioequivalent as defined herein to that prototype formulation.

As in other embodiments, administration according to the present embodiment can be with or without food, i.e., in a non-fasting or fasting condition. It is generally preferred to administer the present compositions to a non-fasting patient.

Further information of relevance to the present invention is available in a recently published article by Tse et al. (2008) Cancer Res. 68:3421-3428 and supplementary data thereto available at Cancer Research Online (cancerres.aacrjournals.org/). This article and its supplementary data are incorporated in their entirety herein by reference.

### EXAMPLES

The following examples are merely illustrative, and do not limit this disclosure in any way. Trademarked ingredients used in the examples can be substituted with comparable ingredients from other suppliers. Where a pre-blended product such as Phosal 50 PG™Phosal 53 MCT™ or Phosal 50 SA+™ is indicated below, its components can, if desired, be added individually rather than in the form of the pre-blended product. Composition of each of Phosal 50 PG™, Phosal 53 MCT™ and Phosal 50 SA+™ is given above. Other trademarked ingredients used in the examples include:

Capmul PG-8™ of Abitec Corp.: propylene glycol monocaprylate;

Cremophor EL™ of BASF: polyoxyl 35 castor oil;

Imwitor 380™ of Sasol GmbH: glyceryl cocoate/citrate/lactate;

Labrasol™ of Gattefossé: caprylocapryl polyoxyglycerides;

Tween 20™ of Uniqema: polysorbate 20 surfactant;

Tween 80™ of Uniqema: polysorbate 80 surfactant.

All ABT-263 amounts, including concentrations and doses, given in the examples are expressed as free base equivalent doses unless expressly stated otherwise. Where ABT-263 is administered as bis-HCl salt, 1.076 mg ABT-263 bis-HCl provides 1 mg ABT-263 free base equivalent.

### Example 1

### Preparation of an Illustrative Liquid Pharmaceutical Composition

Alcohol, dehydrated USP (ethanol) is added to ABT-263 free base in powder form in a 30 ml amber bottle, to disperse the powder. Phosal 53 MCT™ is then added with agitation until the ABT-263 is completely dissolved. The amounts of ABT-263, ethanol and Phosal 53 MCT™ are selected to provide a solution of ABT-263 at a concentration of 25 mg/ml in a Phosal 53 MCT™/ethanol 10:1 carrier.

As an alternative, ABT-263 bis-HCl can be used in place of the ABT-263 free base. The amount of ABT-263 bis-HCl providing 0.25 g ABT-263 free base equivalent is 0.269 g.

### Example 2

### Preparation of an Illustrative Encapsulated Pharmaceutical Composition

The solution prepared in Example 1 is used as a premix for preparing an encapsulated pharmaceutical composition. Soft elastic gelatin capsules are individually filled with 1 ml of the premix, providing 25 mg ABT-263 per capsule. The capsules are filled using a syringe/needle combination and subsequently heat-sealed.

### Example 3

### PK Study of ABT-737 Formulations in Rats

Single-dose pharmacokinetics of ABT-737 solution formulations were evaluated in Sprague-Dawley rats (Charles River; n=3) after a 5 mg/kg oral dose, administered by gavage. Serial heparinized blood samples were obtained from a tail vein of each animal prior to dosing and 0.25, 0.5, 1, 1.5, 2, 3, 4, 6, 8 and 24 hours after administration. Plasma was separated by centrifugation (13,000 rpm for 4 minutes at approximately 4° C.) and ABT-737 was isolated using protein precipitation with acetonitrile.

ABT-737 and an internal standard were separated from each other and from co-extracted contaminants on a 50×3 mm Keystone Betasil CN™ 5 μm column with an acetonitrile/0.1% trifluoroacetic acid mobile phase (50:50 by volume) at a flow rate of 0.7 ml/min. Analysis was performed on a Sciex API3000™ biomolecular mass analyzer with a heated nebulizer interface. ABT-737 and internal standard peak areas were determined using Sciex MacQuan™ software. The plasma drug concentration of each sample was calculated by least squares linear regression analysis (non-weighted) of the peak area ratio (parent/internal standard) of the spiked plasma standards versus concentration. The plasma concentration data were submitted to multi-exponential curve fitting using WinNonlin 3 (Pharsight).

The area under the plasma concentration-time curve from 0 to t hours (time of the last measured plasma concentration) after dosing (AUC₀₋ₜ) was calculated using the linear trapezoidal rule for the plasma concentration-time profiles. The residual area extrapolated to infinity, determined as the final measured plasma concentration (Cₜ) divided by the terminal elimination rate constant (β), was added to AUC₀₋ₜ to produce the total area under the curve (AUC_{0-∞}). The bioavailability was calculated as the dose-normalized AUC_{0-∞} from oral dosing divided by the corresponding value derived from i.v. (intravenous) dosing, administered as a slow bolus to a jugular vein under light ether anesthetic.

Data (means from 3 animals) are shown in Table 1. These data are not illustrative of the present invention, but are included for comparative purposes, ABT-737 being a compound having the formula

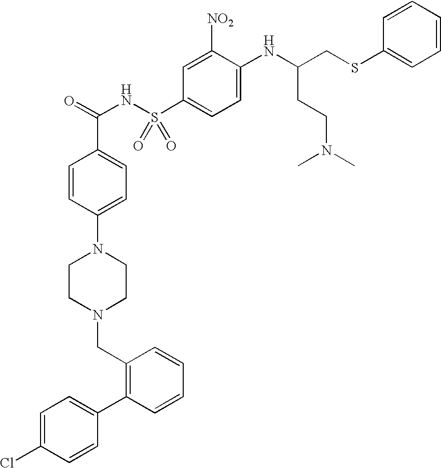

which is closely similar to but not conforming to Formula I.

TABLE 1
PK parameters of ABT-737 solution compositions in rats

| | ABT-737 | | | |
|---|---|---|---|---|
| | conc. | Cₘₐₓ | AUC_{0-∞} | Bioavailability |
| Carrier | (mg/ml) | (μg/ml) | (μg · hr/ml) | (F %) |
| Imwitor 380¹/ethanol² (95:5) | 10 | 0.029 | 0.15 | 3.9 |
| Phosal 53 MCT/ethanol² (90:10) | 10 | 0.028 | 0.13 | 3.3 |
| Phosal 50 PG/ethanol² (90:10) | 50 | 0.024 | 0.06 | 1.5 |
| D5W³/PG⁴/DMSO⁵/Tween⁶ (70:20:5:5) | 5 | 0.032 | 0.23 | 5.9 |

¹unneutralized
²alcohol, dehydrated USP
³dextrose 5% in water
⁴propylene glycol
⁵dimethyl sulfoxide
⁶Tween 20 ™ or Tween 80 ™ can be used

Bioavailability of ABT-737 in rats was extremely low, regardless of the carrier in which the compound was administered.

### Example 4

### PK Study of ABT-263 Free Base Formulations in Rats

Single-dose pharmacokinetics of ABT-263 (free base) solution formulations were evaluated in fasted Sprague-Dawley rats (Charles River; n=3) after a 5 mg/kg oral dose, administered by gavage. Serial heparinized blood samples were obtained from a tail vein of each animal prior to dosing and 0.25, 0.5, 1, 1.5, 2, 3, 4, 6, 8 and 24 hours after administration. Plasma was separated by centrifugation (13,000 rpm for 4 minutes at approximately 4° C.) and ABT-263 was isolated using protein precipitation with acetonitrile. ABT-263 concentrations in plasma were determined and PK parameters calculated as for ABT-737 in Example 3.

Data (means from 3 animals) are shown in Table 2. Data from the PEG 400/DMSO formulation (similar to the formulation reported in above-cited U.S. Patent Application Publication No. 2007/0027135) are not illustrative of the present invention, but are included for comparative purposes.

TABLE 2
PK parameters of ABT-263 solution compositions in rats

| | ABT-263 | | | |
|---|---|---|---|---|
| | conc. | Cₘₐₓ | AUC_{0-∞} | Bioavailability |
| Carrier | (mg/ml) | (μg/ml) | (μg · hr/ml) | (F %) |
| PEG 400/DMSO (90:10) | 2 | 0.67 | 7.53 | 21.6 |
| PEG 400/Phosal 50 PG/DMSO (60:30:10) | 5 | 1.05 | 9.96 | 28.5 |

Bioavailability of ABT-263 compositions in rats was much higher than that of ABT-737 compositions (Example 3). A composition having as carrier a 60:30:10 mixture of PEG 400, Phosal 50 PG and DMSO exhibited higher bioavailability in this rat model than a previously reported composition having as carrier a 90:10 mixture of PEG 400 and DMSO.

### Example 5

### PK Study of ABT-263 Free Base Formulations in Dogs

Single-dose pharmacokinetics of ABT-263 (free base) solution formulations were evaluated in fasted beagle dogs (n=3) after a 2.5, 5 or 10 mg/kg oral dose, administered by gavage followed by 10 ml water. Serial heparinized blood samples were obtained from a jugular vein of each animal prior to dosing and 0.25, 0.5, 1, 1.5, 2, 3, 4, 6, 9, 12, 15 and 24 hours after administration. Plasma was separated by centrifugation (2,000 rpm for 10 minutes at approximately 4° C.) and ABT-263 was isolated using protein precipitation with acetonitrile. ABT-263 concentrations in plasma were determined and PK parameters calculated as in Example 3. The bioavailability was calculated as the dose-normalized AUC_{0-∞} from oral dosing divided by the corresponding value derived from i.v. (intravenous) dosing, administered as a slow bolus to a cephalic vein.

Data (means from 3 animals) are shown in Table 3. Data from the PEG 400/DMSO formulation (similar to the formulation reported in above-cited U.S. Patent Application Publication No. 2007/0027135) are not illustrative of the present invention, but are included for comparative purposes.

TABLE 3
PK parameters of ABT-263 solution compositions in fasted dogs

| | ABT-263 | ABT-263 | | | |
|---|---|---|---|---|---|
| | dose | conc. | Cₘₐₓ | AUC_{0-∞} | Bioavailability |
| Carrier | (mg/kg) | (mg/ml) | (μg/ml) | (μg · hr/ml) | (F %) |
| PEG 400/DMSO (90:10) | 2.5 | 5 | 3.67 | 27.1 | 22.4 |
| | 5 | 10 | 6.75 | 39.8 | 16.5 |
| | 10 | 20 | 8.46 | 58.3 | 12.1 |
| PEG 400/Phosal 50 PG/DMSO | 5 | 10 | 13.22 | 115.1 | 47.6 |
| (60:30:10) | 10 | 20 | 21.6 | 173.9 | 36.0 |

Under fasted conditions, bioavailability of ABT-263 in this dog model was at least 2× greater, dose for dose, when administered in a PEG 400/Phosal 50 PG™/DMSO (60:30:10) carrier than in a PEG 400/DMSO (90:10) carrier.

### Example 6

### Food-Effect PK Study of ABT-263 Free Base Formulation in Dogs

To evaluate food effect, single-dose pharmacokinetics of an ABT-263 (free base) solution formulation of the invention were evaluated in fasted and non-fasted beagle dogs (n=3) after a 10 mg/kg oral dose, administered by gavage followed by 10 ml water. Blood samples were taken, plasma was separated, ABT-263 was isolated, ABT-263 concentrations in plasma were determined and PK parameters were calculated as in Example 5.

Data (means from 3 animals) are shown in Table 4.

TABLE 4
PK parameters of ABT-263 solution compositions in fasted and non-fasted dogs

| | ABT-263 | ABT-263 | | | | |
|---|---|---|---|---|---|---|
| | dose | conc. | | Cₘₐₓ | AUC_{0-∞} | Bioavailability |
| Carrier | (mg/kg) | (mg/ml) | Fasted? | (μg/mL) | (μg · hr/ml) | (F %) |
| PEG 400/Phosal 50 PG/ | 10 | 20 | no | 23.5 | 255.6 | 52.9 |
| DMSO (60:30:10) | | | yes | 20.3 | 156.1 | 32.3 |

Bioavailability of ABT-263 when administered in a PEG 400/Phosal 50 PG™/DMSO (60:30:10) carrier showed a positive food effect in this dog study, non-fasted animals exhibiting higher bioavailability than fasted animals. However, even in fasted animals the bioavailability was >30%. It is believed that the benefit of administering ABT-263 to a non-fasting subject may lie not only in a modest improvement in bioavailability but in a reduced subject-to-subject variability.

### Example 7

### PK Study of ABT-263 Free Base Formulations in Dogs

Single-dose pharmacokinetics of ABT-263 (free base) solution formulations were evaluated in non-fasted beagle dogs (n=3) after a 50 mg/dog oral dose, administered orally in the form of liquid-filled capsules containing approximately 100 mg/ml ABT-263. Additionally, one formulation was tested at a 20 mg/kg oral dose in non-fasted beagle dogs (n=4). Blood samples were taken, plasma was separated, ABT-263 was isolated, ABT-263 concentrations in plasma were determined and PK parameters were calculated as in Example 5.

Data (means from 3 or 4 animals) are shown in Table 5.

TABLE 5
PK parameters of ABT-263 (free base) solution-filled capsule compositions in non-fasted dogs

| | ABT-263 | Cₘₐₓ | AUC_{0-∞} | Bioavailability |
|---|---|---|---|---|
| Carrier | dose | (μg/ml) | (μg · hr/ml) | (F %) |
| PEG 400/Phosal 50 PG/DMSO (60:30:10) | 20 mg/kg | 47.3 | 537.2 | 51.3 |
| Phosal 53 MCT/PEG 400 (70:30) | 50 mg/dog | 21.5 | 119.8 | 53.0 |
| Capmul PG-8/Cremophor EL (90:10) | 50 mg/dog | 10.61 | 63.3 | 27.7 |
| Capmul PG-8 | 50 mg/dog | 6.40 | 59.1 | 24.9 |
| oleic acid/PEG 400/Cremophor EL (80:10:10) | 50 mg/dog | 6.84 | 43.8 | 20.5 |

Compositions of the invention having a carrier comprising Phosal 50 PG™ or Phosal 53 MCT™ exhibited substantially higher ABT-263 bioavailability in this dog model than comparative compositions having different carriers.

### Example 8

### PK Study of ABT-263 bis-HCl Formulations in Dogs

Single-dose pharmacokinetics of ABT-263 bis-HCl solution formulations were evaluated in non-fasted beagle dogs (n=3) after a 46.5 or 50 mg/dog oral dose, administered orally in the form of liquid-filled capsules containing approximately 100 mg/ml ABT-263. Blood samples were taken, plasma was separated, ABT-263 was isolated, ABT-263 concentrations in plasma were determined and PK parameters were calculated as in Example 5.

Data (means from 3 animals) are shown in Table 6.

TABLE 6
PK parameters of ABT-263 bis-HCl solution-filled
capsule compositions in non-fasted dogs

| | ABT-263 | | | |
|---|---|---|---|---|
| | dose | Cₘₐₓ | AUC_{0-∞} | Bioavailability |
| Carrier | (mg/dog) | (μg/ml) | (μg · hr/ml) | (F %) |
| Phosal 53 MCT/PEG 400 (70:30) | 46.5 | 8.89 | 89.6 | 40.8 |
| Labrasol | 46.5 | 8.05 | 65.1 | 29.4 |
| Phosal 53 MCT/Labrasol (70:30) | 50 | 15.83 | 94.3 | 38.5 |
| Labrasol/Tween 20 (70:30) | 50 | 10.31 | 84.8 | 39.0 |
| Phosal 53 MCT/Tween 20 (80:20) | 50 | 14.02 | 89.9 | 48.5 |
| Phosal 53 MCT | 50 | 9.25 | 89.7 | 49.0 |

Compositions of the invention having a carrier comprising Phosal 53 MCT™ all exhibited acceptable ABT-263 bioavailability in this study.

### Example 9

### Phase 1 Clinical PK Study of ABT-263 bis-HCl Formulation

A randomized, placebo-controlled, multi-center, parallel-group study was conducted to evaluate inter alia the PK profile including effect of food on oral bioavailability of an ABT-263 formulation of the present invention in approximately 40 human subjects following dose escalation. The formulation tested was Formulation C as defined herein, prepared from ABT-263 bis-HCl powder dissolved to a concentration of 25 mg/ml in a 90:10 mixture of Phosal 53 MCT™ and dehydrated alcohol USP (ethanol). The formulation was prepared immediately or shortly (not more than about one month) prior to oral administration.

Subjects met all of the following inclusion criteria for participation:
not less than about 18 years old;
a histologically documented diagnosis of a lymphoid malignancy as defined in the WHO classification scheme;
received at least one prior chemotherapy treatment regimen for a lymphoid malignancy and the subject's disease is refractory or the subject had experienced progressive disease following the treatment;
if over the age of 70, had documented brain imaging (MRI or CT) negative for subdural or epidural hematoma within 28 days prior to the first dose of study drug;
an ECOG (Eastern Cooperative Oncology Group) performance score ≦1 (see Table 7 below);
if receiving SSRI anti-depressants, had been receiving a stable dose for at least 21 days prior to the first dose of study drug;
bone marrow ANC (absolute neutrophil count)≧1,000 μl, platelet count ≧100,000/mm³, and hemoglobin level ≧9.0 g/dl;
serum creatinine ≦2.0 mg/dl or calculated creatinine clearance ≧50;
aminotransferases (AST and ALT)≦3×ULN (upper level of normal) and bilirubin ≦1.5×ULN (subjects with Gilbert's syndrome can have bilirubin >1.5×ULN);
coagulation (aPTT and PT) not exceeding 1.2×ULN;
if female, must be surgically sterile, postmenopausal for at least one year or had negative results for a pregnancy test; and
if non-vasectomized male, must have practiced birth control.

The study had several cycles, depending on the subjects' response to the drug. For the first cycle, Formulation C was administered on Day −3 (single day of dosing 3 days prior to Day 1 of Cycle 1), and Days 1-14 followed by seven off-drug days to complete a 24-day cycle (Cycle 1 only). All subjects received Formulation C under fasting conditions on Day −3 and under non-fasting conditions (after a standard breakfast) on Day 1 to study the effect of food on the PK profile of Formulation C. No drug was administered for the 72 hours following the first dose of the first cycle in order to assess the single-dose PK of Formulation C. ABT-263 was administered for 14 consecutive days followed by 7 off-drug days (21-day cycle) for all subsequent cycles. Except for Days −3 and 1 of the first cycle, subjects self-administered ABT-263 orally once daily (q.d.) approximately 30 minutes after a breakfast. providing approximately 520 Kcal, with approximately 30% calories from fat.

Formulation C dosing began at 10 mg ABT-263 and escalated to a maximum tolerated dose (MTD) with at least 3 subjects in each cohort. The dose doubled until one grade 3 or two grade 2 toxicities occurred, after which dose escalated in 25-40% increments. Platelet levels were monitored and reviewed to inform dose escalation decisions.

The first subject in each cohort completed two weeks of dosing before more subjects enrolled. Escalation to the next dose level proceeded when all assigned subjects in a given cohort completed the cycle without experiencing a dose-limiting toxicity (DLT). If one subject within any dose level experienced a DLT, a total of 6 subjects were enrolled at that dose level.

A physical examination, including weight, oral body temperature, blood pressure, and pulse, was performed at Screening, Cycle 1 Day −3, Day 1 of each subsequent cycle (or within 72 hours prior), and the Final Visit. A symptom-directed physical examination was performed weekly through the first 2 cycles and whenever necessary. The ECOG performance status (Table 7) was assessed at screening, Cycle 1 Day −3, lead-in Day 1, weekly through the first two cycles, Day 1 of each subsequent cycle (or within 72 hours prior), a final visit, and a safety follow-up visit.

TABLE 7
ECOG performance grades and descriptions

| Grade | Description |
|---|---|
| 0 | Fully active, able to carry on all pre-disease performance without restriction. |
| 1 | Restricted in physically strenuous activity but ambulatory and able to carry out |
| | work of a light or sedentary nature, e.g., light housework, office work |
| 2 | Ambulatory and capable of all self-care but unable to carry out any work |
| | activities. Up and about more than 50% of waking hours. |
| 3 | Capable of only limited self-care, confined to bed or chair more than 50% of |
| | waking hours. |
| 4 | Completely disabled. Cannot carry on any self-care. Totally confined to bed or |
| | chair. |

Blood and plasma samples were protected from direct sunlight during collection, processing and storage. The timing of blood collections took priority over other scheduled study activities except for dosing. The order of blood collections was maintained to the minute such that the time intervals relative to the preceding dosing were the same for all subjects.

Blood samples were collected by venipuncture into 3-ml evacuated potassium EDTA-containing collection tubes during Cycle 1 Day −3, prior to dosing (0 hour) and at 0.5, 1, 2, 3, 4, 6, 8, 24, 48 and 72 (Day 1, predose sample) hours after dosing; Day 1, at 0.5, 1, 2, 3, 4, 6, 8 and 24 (Day 2, pre-dose sample) hours after dosing; Day 14, prior to dosing (0 hour) and at 0.5, 1, 2, 3, 4, 6 and 8 hours after dosing. Additional blood samples were collected at 0 hour (pre-dose) on Day 14, Cycle 2 through Cycle 6. Sufficient blood was collected to provide approximately 1 ml plasma from each sample. A total of 27 blood samples (approximately 81 ml) were collected per subject for pharmacokinetic analysis during Cycle 1 and one additional blood sample per subject per cycle, up to Cycle 6.

Values for the pharmacokinetic parameters of ABT-263, including maximum observed plasma concentration (Cₘₐₓ), time to Cₘₐₓ(peak time, Tₘₐₓ), terminal phase elimination rate constant (f₃), terminal elimination half-life (t_{1/2}), area under the plasma concentration-time curve (AUC) from time 0 to the time of the last measurable concentration (AUC₀₋ₜ), e.g., from time 0 to 24 hours (AUC₀₋₂₄), and from time 0 to infinite time (AUC_{0-∞}) for the doses on Cycle 1 Day −3, Cycle 1 Day 1 and Cycle 1 Day 14 whenever applicable, were determined using noncompartmental methods.

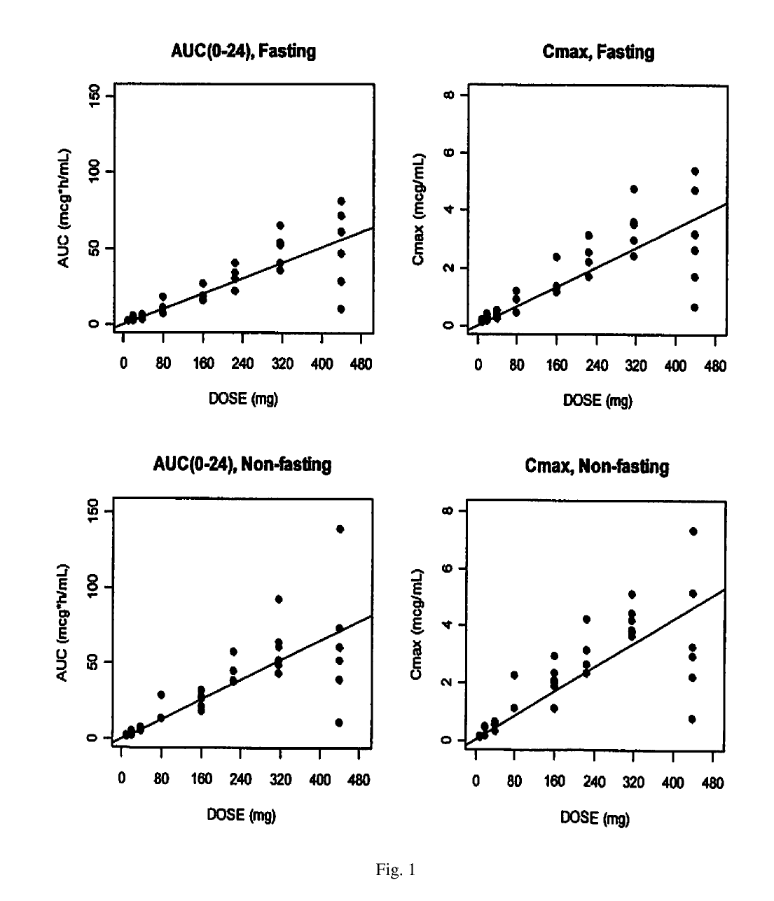

As shown in FIG. 1, the human PK parameters Cₘₐₓ and AUC₀₋₂₄ on single dosing were found to be substantially dose-proportional in this study, at least up to the 315 mg dose. This was true under both fasting (Day −3) and non-fasting (Day 1) conditions. The difference between fasting and non-fasting conditions in Cₘₐₓ and AUC₀₋₂₄ was minor, showing only a mild positive food effect on ABT-263 absorption following oral administration of Formulation C.

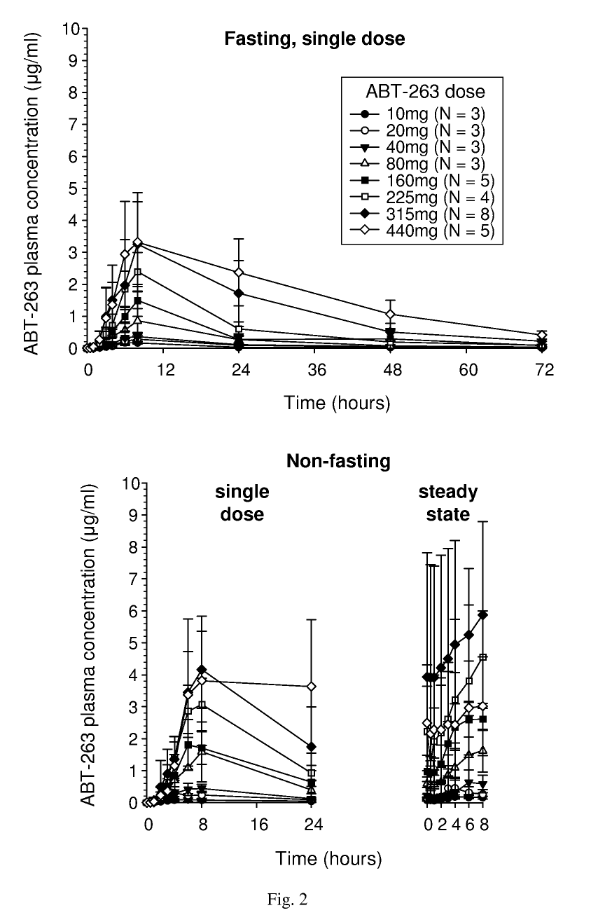

As shown in FIG. 2, Tₘₐₓ was around 8 hours in both fasting and non-fasting conditions. Upon daily dosing at 315 mg/day, plasma concentration of ABT-263 at steady state (Day 14) was about 3 μg/ml (trough) and about 5.5 μg/ml (peak).

PK parameters for single-dose fasting, single-dose non-fasting and steady-state non-fasting (Days −3, 1 and 14 respectively) at a range of ABT-263 doses are presented in Tables 8, 9 and 10 below.

It is believed that a therapeutically effective daily dose of ABT-263 administered orally in Formulation C is about 200 to about 400 mg for most patients, providing a steady-state Cₘₐₓ of about 4 to about 7 μg/ml.

TABLE 8
PK parameters of ABT-263 bis-HCl (Formulation C) in humans
(single-dose, fasting)

| Dose | | Tₘₐₓ | Cₘₐₓ | AUC₀₋₂₄ | AUC_{0-∞} |
|---|---|---|---|---|---|
| (mg) | N | (h) | (μg/ml) | (μg · hr/ml) | (μg · hr/ml) |
| 10 | 3 | 6.7 ± 1.2 | 0.18 ± 0.05 | 2.2 ± 0.4 | 3.2 ± 1.1 |
| 20 | 3 | 7.3 ± 1.2 | 0.29 ± 0.13 | 3.9 ± 1.8 | 6.4 ± 3.1 |
| 40 | 3 | 7.3 ± 1.2 | 0.37 ± 0.13 | 5.0 ± 1.6 | 8.4 ± 3.4 |
| 80 | 3 | 8.0 ± 0.0 | 0.85 ± 0.39 | 11.9 ± 5.8 | 17.6 ± 9.3 |
| 160 | 5 | 8.0 ± 0.0 | 1.5 ± 0.5 | 18.8 ± 4.4 | 32.5 ± 7.8 |
| 225 | 4 | 7.5 ± 1.0 | 2.4 ± 0.6 | 31.7 ± 7.8 | 46.5 ± 12.4 |
| 315 | 8 | 11.8 ± 7.6 | 3.6 ± 1.1 | 50.5 ± 15.6 | 91.0 ± 33.5 |
| 440 | 6 | 10.0 ± 6.9 | 3.0 ± 1.8 | 49.7 ± 27.1 | 109.7 ± 53.8 |

TABLE 9
PK parameters of ABT-263 bis-HCl (Formulation
C) in humans (single-dose, non-fasting)

| Dose | | Tₘₐₓ | Cₘₐₓ | AUC₀₋₂₄ |
|---|---|---|---|---|
| (mg) | N | (h) | (μg/ml) | (μg · hr/ml) |
| 10 | 3 | 11.3 ± 11 | 0.12 ± 0.03 | 1.5 ± 0.5 |
| 20 | 3 | 4.0 ± 1.7 | 0.36 ± 0.18 | 4.2 ± 2.1 |
| 40 | 3 | 6.0 ± 2.0 | 0.50 ± 0.15 | 6.4 ± 1.2 |
| 80 | 2 | 7.0 ± 1.4 | 1.7 ± 0.8 | 21.1 ± 10.7 |
| 160 | 6 | 9.7 ± 7.1 | 2.1 ± 0.6 | 25.9 ± 5.5 |
| 225 | 4 | 7.5 ± 1.0 | 3.1 ± 0.8 | 43.9 ± 9.5 |
| 315 | 9 | 7.6 ± 1.0 | 4.4 ± 1.1 | 58.5 ± 17.5 |
| 440 | 6 | 15.3 ± 9.5 | 3.6 ± 2.3 | 62.2 ± 43.2 |

TABLE 10
PK parameters of ABT-263 bis-HCl (Formulation
C) in humans (steady-state, non-fasting)

| Dose | | Tₘₐₓ | Cₘₐₓ |
|---|---|---|---|
| (mg/day) | N | (h) | (μg/ml) |
| 10 | 3 | 5.0 ± 2.6 | 0.19 ± 0.06 |
| 20 | 3 | 4.3 ± 1.5 | 0.48 ± 0.31 |
| 40 | 3 | 6.7 ± 1.2 | 0.65 ± 0.37 |
| 80 | 3 | 7.3 ± 1.2 | 1.8 ± 1.0 |
| 160 | 5 | 6.0 ± 1.4 | 2.8 ± 0.5 |
| 225 | 3 | 7.3 ± 1.2 | 4.6 ± 1.6 |
| 315 | 8 | 5.3 ± 2.8 | 6.4 ± 3.2 |
| 440 | 6 | 4.2 ± 3.7 | 3.0 ± 1.5 |

### Example 10

### Clinical PK Study of ABT-263 Formulations in Healthy Human Subjects

A Phase 1, single dose, open-label study was conducted according to a three-period, randomized, crossover design to evaluate the PK profile of ABT-263 solution formulations of the present invention in healthy female subjects (n=12) of non-childbearing potential (surgically sterile or post-menopausal) at a single dose of 25 mg ABT-263 free base equivalent.

ABT-263 free base was dissolved to a concentration of 25 mg/ml or 50 mg/ml (Formulations B1 and B2 respectively) in a carrier consisting of a 90:10 v/v mixture of Phosal 53 MCT™ and dehydrated alcohol USP (ethanol). It will be noted that the carrier in Formulations B1 and B2 is identical to that used in Formulation C, which contains ABT-263 bis-HCl rather than ABT-263 free base (see Example 9 above). The oral bioavailability of Formulations B1 and B2 was compared with that of Formulation C.

Unmixed ABT-263 free base or ABT-263 bis-HCl powders were stored at 15-25° C. with protection from light. The formulations were prepared by dissolution of the appropriate powder in the carrier at the required concentration immediately or shortly (not more than one month) prior to oral administration. Once so constituted, the formulations, unless administered immediately upon preparation, were stored at 2-8° C. with protection from light.

A total of 12 subjects were randomly assigned in equal numbers to Sequences I, II, and III (see Table 11). Each sequence consisted of three periods. Subjects were confined to the study site and supervised for a minimum of 17 days beginning one day before administration of ABT-263 in Period 1 (Day −1) and ending after completion of all study procedures at the end of Period 3.

TABLE 11
Study design

| Sequence | n* | Period 1 | Period 2 | Period 3 |
|---|---|---|---|---|
| I | 4 | Formulation C | Formulation B1 | Formulation B2 |
| II | 4 | Formulation B1 | Formulation B2 | Formulation C |
| III | 4 | Formulation B2 | Formulation C | Formulation B1 |

*evaluable subjects

Blood samples were collected by venipuncture into 3 ml evacuated collection tubes containing potassium EDTA during each period at 0 hour (pre-dose) and 2, 4, 6, 8, 10, 12, 14, 16, 24, 30, 48 and 72 hours (post-dose). Sufficient blood was collected to provide approximately 1.5 ml plasma from each sample.

Blood samples were centrifuged within one hour of collection using a refrigerated centrifuge (2-8° C.) to separate plasma. The resulting plasma samples were transferred using plastic pipettes into labeled, screw-capped polypropylene tubes, were frozen at −20° C. or colder within one hour after collection and remained frozen until analysis. A maximum of 32 days elapsed between collection and analysis.

Plasma concentrations of ABT-263 were determined using a validated liquid chromatography method with Tandem Mass Spectrometric detection. All three formulations for each subject were analyzed in the same analytical run. PK parameters for Formulations C, B1 and B2 are presented in Table 12 below.

The Cₘₐₓ and AUC_{0-∞} for the 25 mg/ml ABT-263 free base formulation (Formulation B1) were about 106% and 101%, respectively, of the values for Formulation C. The Cₘₐₓ and AUC_{0-∞} for the 50 mg/ml ABT-263 free base formulation (Formulation B2) were about 95% and 98%, respectively, of the values for Formulation C.

TABLE 12
PK parameters of ABT-263 solution formulations in healthy human subjects
(single 25 mg dose)

| | ABT-263 | | | | AUC₀₋₇₂ | AUC_{0-∞} |
|---|---|---|---|---|---|---|
| Formulation | form | Concentration | Cₘₐₓ (μg/ml) | Tₘₐₓ (h) | (μg · hr/ml) | (μg · hr/ml) |
| C | bis-HCl | 25 mg/ml | 0.68 ± 0.15 | 6.2 ± 0.6 | 8.32 ± 2.07 | 8.67 ± 2.19 |
| B1 | free base | 25 mg/ml | 0.72 ± 0.14 | 6.7 ± 1.8 | 8.44 ± 2.07 | 8.80 ± 2.23 |
| B2 | free base | 50 mg/ml | 0.64 ± 0.11 | 7.5 ± 2.7 | 8.16 ± 2.25 | 8.52 ± 2.36 |

### Example 11

### Clinical PK Study of ABT-263 Formulations in Human Cancer Patients

A cross-over study was conducted to evaluate the PK profile of ABT-263 formulations of the present invention (Formulations C and B1 as used in Example 10 above) in 12 human cancer patients at a single dose of 250 mg ABT-263 free base equivalent. The formulations were prepared immediately or shortly (not more than one month) prior to oral administration.

A total of 13 subjects were enrolled in Sequences I and II (see Table 13), of which 12 completed both periods. One subject completed Period 1 only and was excluded from analysis. Blood samples were collected by venipuncture prior to formulation administration (0 hour) and at 2, 4, 6, 8, 10, 12, 24, 30 and 48 hours after dosing.

TABLE 13
Study design

| Sequence | n* | Period 1 | Period 2 |
|---|---|---|---|
| I | 6 | Formulation C | Formulation B1 |
| II | 6 | Formulation B1 | Formulation C |

*evaluable subjects

PK parameters for Formulations C and B1 are presented in Table 14 below. Nine subjects displayed similar bioavailability of the ABT-263 free base solution (Formulation B1) and the ABT-263 bis-HCl solution (Formulation C). The remaining 3 subjects displayed relatively high bioavailability of Formulation B1. The Cₘₐₓ and AUC values for these patients were still within the range of exposure seen in other patients.

TABLE 14
PK parameters of ABT-263 solution formulations in human cancer patients
(single 250 mg dose)

| | ABT-263 | | | | AUC₀₋₄₈ | AUC_{0-∞} |
|---|---|---|---|---|---|---|
| Formulation | form | Concentration | Cₘₐₓ (μg/ml) | Tₘₐₓ (h) | (μg · hr/ml) | (μg · hr/ml) |
| C | bis-HCl | 25 mg/ml | 2.98 ± 1.30 | 9.2 ± 1.6 | 61.1 ± 29.6 | 70.3 ± 34.8 |
| B1 | free base | 25 mg/ml | 3.82 ± 1.58 | 9.8 ± 1.6 | 75.3 ± 31.1 | 83.4 ± 35.2 |

What is claimed is:

1. An orally deliverable pharmaceutical composition comprising a drug-carrier system that comprises a compound of Formula I: 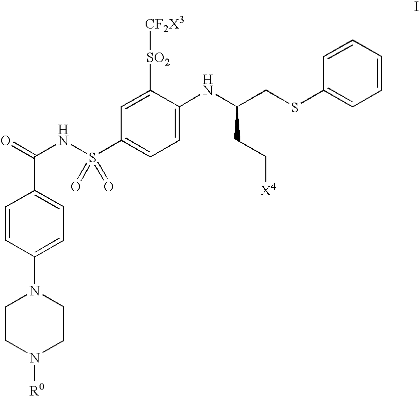
where X³ is chloro or fluoro; and
(1) X⁴ is azepan-1-yl, morpholin-4-yl, 1,4-oxazepan-4-yl, pyrrolidin-1-yl, N(CH₃)₂, N(CH₃)(CH(CH₃)₂), 7-azabicyclo[2.2.1]heptan-1-yl or 2-oxa-5-azabicyclo[2.2.1]hept-5-yl; and R⁰ is 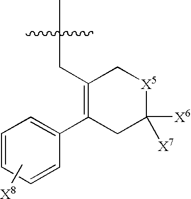
where
X⁵ is CH₂, C(CH₃)₂ or CH₂CH₂;
X⁶ and X⁷ are both hydrogen or both methyl; and
X⁸ is fluoro, chloro, bromo or iodo; or
(2) X⁴ is azepan-1-yl, morpholin-4-yl, pyrrolidin-1-yl, N(CH₃)(CH(CH₃)₂) or 7-azabicyclo[2.2.1]heptan-1-yl; and R⁰ is 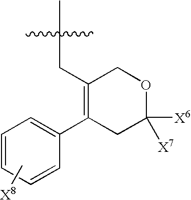
where X⁶, X⁷ and X⁸ are as above; or
(3) X⁴ is morpholin-4-yl or N(CH₃)₂; and R⁰ is 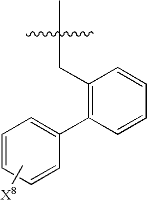
where X⁸ is as above;
or a pharmaceutically acceptable salt, prodrug, salt of a prodrug or metabolite thereof; in solution in a substantially non-aqueous carrier that comprises a phospholipid component and a pharmaceutically acceptable solubilizing component; wherein said carrier comprises zero to about 25% by weight ethanol.

2. The composition of claim 1, wherein, in the compound of Formula I, X³ is fluoro.

3. The composition of claim 1, wherein, in the compound of Formula I, X⁴ is morpholin-4-yl.

4. The composition of claim 1, wherein, in the compound of Formula I, R⁰ is 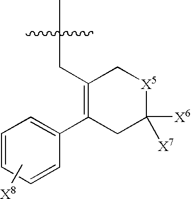
where
X⁵ is O, CH₂, C(CH₃)₂ or CH₂CH₂;
X⁶ and X⁷ are both hydrogen or both methyl; and
X⁸ is fluoro, chloro, bromo or iodo.

5. The composition of claim 1, wherein, in the compound of Formula I, R⁰ is 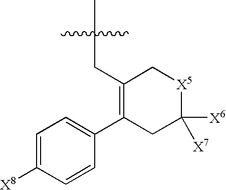
where
X⁵ is O, CH₂, C(CH₃)₂ or CH₂CH₂;
X⁶ and X⁷ are both hydrogen or both methyl; and
X⁸ is fluoro, chloro, bromo or iodo.

6. The composition of claim 5, wherein, in the compound of Formula I, X⁵ is CH₂ or C(CH₃)₂ and/or each of X⁶ and X⁷ is methyl and/or X⁸ is chloro.

7. The composition of claim 1, wherein the compound of Formula I is ABT-263 or a salt, prodrug, salt of a prodrug or metabolite thereof.

8. The composition of claim 7, wherein said compound is ABT-263 free base or ABT-263 bis-HCl.

9. The composition of claim 8, wherein the drug-carrier system is liquid.

10. The composition of claim 9, wherein the compound is present in an amount of about 10 to about 500 mg/ml free base equivalent.

11. The composition of claim 9, wherein the phospholipid component of the carrier comprises phosphatidylcholine.

12. The composition of claim 9, wherein the solubilizing component of the carrier comprises one or more glycols, glycolides and/or glyceride materials.

13. The composition of claim 9, wherein the solubilizing component of the carrier comprises one or more medium chain triglycerides.

14. The composition of claim 9, wherein the carrier comprises about 15% to about 75% by weight phosphatidylcholine, about 5% to about 70% by weight of one or more glyceride materials, 0% to about 25% ethanol and 0% to about 5% surfactant.

15. The composition of claim 9, wherein the carrier comprises about 3% to about 15% by weight ethanol.

16. The composition of claim 9, wherein the ABT-263 free base or ABT-263 bis-HCl is present in an amount of about 20 to about 200 mg/ml free base equivalent.

17. The composition of claim 16, wherein the carrier is selected to provide oral bioavailability of ABT-263 of at least about 30% when the composition is administered as a single dose of about 2.5 to about 10 mg/kg in a fasting or non-fasting dog model.

18. The composition of claim 7 that is
(a) a prototype formulation comprising ABT-263 bis-HCl in a free base equivalent amount of about 25 mg/ml, in solution in a carrier that comprises (i) about 90% of a product comprising about 53% by weight phosphatidylcholine and about 29% by weight medium chain triglycerides, and (ii) about 10% ethanol; or
(b) a formulation that is orally substantially bioequivalent to said prototype formulation.

19. The composition of claim 7 that is
(a) a prototype formulation comprising ABT-263 free base in an amount of about 25 to about 50 mg/ml, in solution in a carrier that comprises (i) about 90% of a product comprising about 53% by weight phosphatidylcholine and about 29% by weight medium chain triglycerides, and (ii) about 10% ethanol; or
(b) a formulation that is orally substantially bioequivalent to said prototype formulation.

20. A method for treating a disease characterized by apoptotic dysfunction and/or overexpression of an anti-apoptotic Bcl-2 family protein, comprising orally administering to a subject having the disease a therapeutically effective amount of the composition of claim 1.

21. The method of claim 20, wherein the disease is a neoplastic disease.

22. The method of claim 21, wherein the neoplastic disease is selected from the group consisting of cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma and combinations thereof.

23. The method of claim 21, wherein the neoplastic disease is a lymphoid malignancy.

24. The method of claim 23, wherein the lymphoid malignancy is non-Hodgkin's lymphoma.

25. The method of claim 21, wherein the neoplastic disease is chronic lymphocytic leukemia or acute lymphocytic leukemia.

26. The method of claim 20, wherein the composition administered comprises ABT-263 or a salt, prodrug, salt of a prodrug or metabolite thereof.

27. The method of claim 26, wherein the composition administered comprises ABT-263 free base or ABT-263 bis-HCl.

28. The method of claim 26, wherein the composition is administered in a dose of about 50 to about 500 mg ABT-263 free base equivalent per day at an average treatment interval of about 3 hours to about 7 days.

29. The method of claim 26, wherein the composition is administered once daily in a dose of about 200 to about 400 mg ABT-263 free base equivalent per day.

30. The method of claim 26, wherein the composition administered is
(a) a prototype formulation comprising ABT-263 bis-HCl in a free base equivalent amount of about 25 mg/ml, in solution in a carrier that comprises (i) about 90% of a product comprising about 53% by weight phosphatidylcholine and about 29% by weight medium chain triglycerides, and (ii) about 10% ethanol; or
(b) a formulation that is orally substantially bioequivalent to said prototype formulation.

31. The method of claim 26, wherein the composition administered is
(a) a prototype formulation comprising ABT-263 free base in an amount of about 25 to about 50 mg/ml, in solution in a carrier that comprises (i) about 90% of a product comprising about 53% by weight phosphatidylcholine and about 29% by weight medium chain triglycerides, and (ii) about 10% ethanol; or
(b) a formulation that is orally substantially bioequivalent to said prototype formulation.

32. A method for maintaining in bloodstream of a human subject a therapeutically effective plasma concentration of ABT-263 and/or one or more metabolites thereof, comprising administering to the subject a pharmaceutical composition comprising a drug-carrier system that comprises ABT-263 or a pharmaceutically acceptable salt, prodrug, salt of a prodrug or metabolite thereof, in solution in a substantially non-aqueous carrier that comprises a phospholipid component and a pharmaceutically acceptable solubilizing component, in a dosage amount of about 50 to about 500 mg ABT-263 free base equivalent per day, at an average dosage interval of about 3 hours to about 7 days.

33. The method of claim 32, wherein the plasma concentration maintained exhibits, at steady state, a peak of about 3 to about 8 μg/ml ABT-263 and a trough of about 1 to about 5 μg/ml ABT-263.

34. The method of claim 32, wherein the composition is administered once daily in a dose of about 200 to about 400 mg ABT-263 free base equivalent per day, said composition being
(a) a prototype formulation comprising ABT-263 bis-HCl in a free base equivalent amount of about 25 mg/ml, in solution in a carrier that comprises (i) about 90% of a product comprising about 53% by weight phosphatidylcholine and about 29% by weight medium chain triglycerides, and (ii) about 10% ethanol; or
(b) a formulation that is orally substantially bioequivalent to said prototype formulation.

35. The method of claim 32, wherein the composition is administered once daily in a dose of about 200 to about 400 mg ABT-263 free base equivalent per day, said composition being
(a) a prototype formulation comprising ABT-263 free base in an amount of about 25 to about 50 mg/ml, in solution in a carrier that comprises (i) about 90% of a product comprising about 53% by weight phosphatidylcholine and about 29% by weight medium chain triglycerides, and (ii) about 10% ethanol; or
(b) a formulation that is orally substantially bioequivalent to said prototype formulation.
